(19) United States
(12) Patent Application Publication
(10) Pub. No.: US 2024/0147927 A1
(43) Pub. Date: May 9, 2024

(54) METHODS OF HIGH PRODUCTION OF POLYPHENOLS FROM RED LETTUCES AND USES THEREOF

(71) Applicant: SignalChem Plantech Corporation, Richmond (CA)

(72) Inventors: Hao Chen, Richmond (CA); Tiehan Zhao, Richmond (CA); Xiaohui Yao, Richmond (CA); Zaihui Zhang, Richmond (CA); Jun Yan, Richmond (CA)

(21) Appl. No.: 18/547,845

(22) PCT Filed: Feb. 25, 2022
(86) PCT No.: PCT/US2022/017940

§ 371 Date: Aug. 24, 2023

Related U.S. Application Data
(60) Provisional application No. 63/154,529, filed on Feb. 26, 2021.

(51) Int. Cl. A01H 3/04; A61K 36/28; A61P 29/00; A61P 31/14; A61P 31/16; A61P 35/00

(52) U.S. Cl. CPC A01H 3/04; A61K 36/28; A61P 29/00; A61P 31/14; A61P 31/16; A61P 35/00; A61K 2236/33

(57) ABSTRACT

Provided herein are systems and methods for enhancement of polyphenols, such as chlorogenic acids, chicoric acid, anthocyanins, and water-soluble quercetin derivatives, production in red lettuces. Also provided are transgenic lettuce for the production of polyphenols. Also provided are parts of such transgenic lettuces, such as seeds leaves, and extracts. The disclosure also provides methods of using the new lettuces and parts thereof for protection against viral/bacterial infection (i.e., by inhibiting activities of COVID-19 virus/enzymes) diabetes, cardiovascular diseases, memory and eyesight loss, inflammation, and cancer.

### BACKGROUND

Polyphenols, such as water-soluble quercetin derivatives, chicoric acid, chlorogenic acids, and anthocyanins, are beneficial plant compounds with antioxidant properties that may help keep one healthy and protect against various diseases. There is an increasing demand by consumers for nutritious foods that improve physical performance, reduce risks of disease, and increase life span. Researchers and food manufacturers are interested in increasing health beneficial polyphenols in foods, due to the antioxidant properties of these compounds and their role in the prevention of various diseases, such as many types of cancer, cardiovascular and neurodegenerative diseases. Since these health-promoting effects depend on relatively high level of polyphenols, there is a strong need to increase their amounts in human diet. Although blueberries are one of the richest sources of polyphenols and are highly recommended for human consumption, their consumption per capita is still low compared to other types of fresh fruits and vegetables. Moreover, blueberries contain high amounts of sugar, which may not be desirable for many individuals. Thus, there is a need to develop other plants with increased health beneficial polyphenol content, with less sugar that could gain wide popularity among public, and can become part of everyday food intake.

Lettuce (Lactuca sativa L.) is widely used in salads and sandwiches, and is an important component in human diet and nutrition. Recently, lettuce was the second most consumed fresh vegetable in the USA. Thus, novel red lettuces that can produce high content of polyphenols may be both commercially viable and health beneficial.

### BRIEF SUMMARY

The present disclosure provides red lettuces with significantly increased amounts of health beneficial polyphenols such as quercetin derivatives, chicoric acid, chlorogenic acids, and anthocyanins. Also provided herein are methods of producing such red lettuces, for example, by (1) using plant eustressors/elicitors to stimulate the production of desired secondary metabolites as well as (2) regulating genes of the phenylpropanoid pathway to enhance the downstream secondary metabolites. The disclosure also provides extracts from such lettuces, methods of making such extracts, and methods of using such extracts, for example, to inhibit viral replication, reduce inflammation, improve visual acuity, modulate the immune response, reduce obesity and diabetes, reduce blood glucose levels, or combinations thereof.

In some embodiments, disclosed herein is a system for biosynthesis of polyphenols in lettuce that comprises at least one elicitor, or a homologue, isomer or derivative thereof that increase the production of polyphenols in lettuce.

In some embodiments, disclosed herein is a system for biosynthesis of polyphenols in lettuce that comprises an expression cassette comprising a heterologous expression control sequence operably linked to at least one polynucleotide encoding one or more proteins that increase the production of polyphenols in lettuce.

In some embodiments, disclosed herein is a system for increasing production of polyphenols in lettuce that comprises the at least one elicitor, or a homologue, isomer or derivative thereof of the present disclosure and the expression cassette of the present disclosure.

These and other aspects of the present disclosure will become apparent upon reference to the following detailed description and attached drawings. All references disclosed herein are hereby incorporated by reference in their entirety as if each was incorporated individually.

### BRIEF DESCRIPTION OF THE SEVERAL VIEWS OF THE DRAWINGS

FIGS. 1A-1B shows HPLC-UV chromatograms of bioactive components enhancement by genomics-based technologies confirming production of specific metabolites from red lettuce treated with eustressor/elicitors. FIG. 1A shows non-treated lettuce. FIG. 1B shows treated lettuce: A: Chlorogenic acid (3-CQA); B: Chicoric acid (CRA); C: Quercetin-3-O-glucoside (Q3G); D: Quercetin-3-O-malonylglucoside (Q3MG); E: 3,4-Dicaffeoylquinic acid (3,4-diCQA)

FIGS. 2A-2B show the production of chlorogenic acids and chicoric acid and the water-soluble quercetin derivatives were increased by 3- to 9-fold in red lettuce treated with plant growth regulators. FIG. 2A depicts production of chlorogenic acid, 3,4-dicaffeoylquinic acid (3,4-diCQA), and chicoric acid (3-CQA, CRA, and 3,4-diCQA). FIG. 2B depicts production of quercetin derivatives (Q3G and Q3MG).

FIGS. 3A-3B shows HPLC-UV chromatograms of bioactive components enhancement by genomics-based technologies confirming production of specific metabolites from red lettuce treated by regulation of genes of the phenylpropanoid pathway. FIG. 1A shows non-treated lettuce. FIG. 1B shows treated lettuce: A: Chlorogenic acid (3-CQA); B: Chicoric acid (CRA); C: Quercetin-3-O-glucoside (Q3G); D: Quercetin-3-O-malonylglucoside (Q3MG); E: 3,4-Dicaffeoylquinic acid (3,4-diCQA)

FIGS. 4A-4B show levels of phenylpropanoid pathway products in treated lettuce and untreated control. FIG. 4A shows the production of chlorogenic acids. FIG. 4B shows the production of water-soluble quercetin derivatives.

FIG. 5 shows inhibition of SARS-CoV-2 3-chymotrypsin-like protease (3CL^{pro}). Much stronger inhibitory effect of SLC1021 (Red lettuce extract) (3CL^{pro}+SLC1021) was demonstrated when compared to the untreated plant extract (3CL^{pro}+control) or pure quercetin-3-O-glucoside (3CL^{pro}+Q3G).*: equivalent to 100 mM of quercetin derivatives in the plant extract.

FIG. 6 shows inhibition of SARS-CoV-2 RNA-dependent RNA polymerase (RdRp). Stronger inhibitory effect of SLC1021 (RdRp+SLC1021) was observed when compared to the untreated plant extract (RdRp+control) & metabolized remdesivir (RdRp+RTP).*: equivalent to 100 mM of quercetin derivatives in the plant extract.

FIG. 7 shows inhibition of SARS-CoV-2 RNA helicase and triphosphatase (nsp13). Stronger inhibitory effect of SLC1021 (nsp13+SLC1021) was observed when compared to untreated plant extract (nsp13 +control)*: equivalent to 100 mM of quercetin derivatives in the plant extract.

FIG. 8 shows results of red lettuce extract SLC1021 on in vitro SARS-CoV2 infection induced cytopathic effect (CPE) in Vero E6 cells.

FIG. 9 shows blocking of 2019-nCoV Spike protein receptor binding domain (RBD) binding of ACE2-CHO cells by red lettuce extract SLC1021 at 10 μg/mL and 100 μg/mL. 10 μg/mL of Spike protein was used as a negative control. The binding was determined anti-Spike protein antibody staining and fluorescence flow cytometry.

FIGS. 10A-10B shows red lettuce extract SLC1021 in vitro inhibition of cytopathic effect by Influenza virus A (Flu A) and respiratory syncytia virus (RSV). FIG. 10A shows SLC1021 inhibition of the cytopathic effect cause by Flu A. FIG. 10B shows SLC1021 inhibition of the cytopathic effect cause by RSV. The percent reduction in viral CPE and the percent of cell control was determined from cells without SLC1021 treatment.

FIG. 11 shows the results of MTS assays performed with Jurkat, HL60, THP1, MCF7 and LNCaP cells treated with increasing concentrations of SLC1021 compared to untreated control cells. The data are presented as mean±SE. % of cell control was determined from cells without treatment.

FIG. 12 shows the effect of SLC1021 on reactive oxygen species (ROS) in Jurkat cells and human primary T-cells as assessed by detection of DCF-DA fluorescence using flow cytometry. The data are presented as the ratio of the mean fluorescence intensity (MFU) comparing SLC1021 treated cells to untreated control.

FIGS. 13A-13F show results of comparison studies assessing the cytotoxic effect of SLC1021, SLC1021-B, 4-CQA, neochlorogenic acid, chicoric acid, and cyanidin 3-galactoside on Jurkat, THP1 and MCF7 cancer cells as determined by MTS assay. FIG. 13A shows results of treatment with SLC1021. FIG. 13B shows results of treatment with SLC1021-B. FIG. 13C shows results of treatment with 4-CQA. FIG. 13D shows results of treatment with neochlorogenic acid. FIG. 13E shows results of treatment with chicoric acid. FIG. 13F shows results of treatment with cyanidin 3-galactoside. The data are presented as mean±SE. Percent (%) of cell control was determined from untreated control cells.

FIG. 14 shows an anti-inflammatory effect of SLC1021 on IL-6 and TNFα production in LPS treated macrophages. The production of cytokines was measured by ELISA. The data are presented as mean±SE. Percent (%) of control was determined from LPS-treated macrophages without SLC1021.

FIG. 15 shows the anti-inflammatory effect of SLC1021-B on IL-6 and TNFα production in LPS treated macrophages. The production of cytokines was measured by ELISA. The data are presented as mean±SE. Percent (%) of control was determined from LPS-treated macrophages without SLC1021-B.

FIGS. 16A-16D show the effect of 4-CQA, neochlorogenic acid, chicoric acid, and cyanidin 3-galactoside on IL-6 and TNF-α production on LPS-induced macrophages. FIG. 16A shows cytokine production in 4-CQA treated cells. FIG. 16B shows cytokine production in neochlorogenic acid treated cells. FIG. 16C shows cytokine production in chicoric acid treated cells. FIG. 16D shows cytokine production in cyanidin 3-galactoside treated cells. The production of cytokines was measured by ELISA. The data are presented as mean±SE. Percent (%) of control was determined from LPS treated cells without test agent treatment.

FIG. 17 shows an anti-oxidant effect of SLC1021 on nitric oxide production in LPS treated macrophages. The production of nitric oxide was measured by ELISA. The data are presented as mean±SE. Percent (%) of control was determined from LPS-treated macrophages without SLC1021.

FIG. 18 shows an anti-oxidant effect of SLC1021-B on nitric oxide production in LPS treated macrophages. The production of nitric oxide was measured by ELISA. The data are presented as mean±SE. Percent (%) of control was determined from LPS-treated macrophages without SLC1021-B.

FIGS. 19A-19D show the effect of 4-CQA, neochlorogenic acid, chicoric acid, and cyanidin 3-galactoside on nitric oxide (NO) production on LPS-induced macrophages. FIG. 19A shows NO production in 4-CQA treated cells. FIG. 19B shows NO production in neochlorogenic acid treated cells. FIG. 19C shows NO production in chicoric acid treated cells. FIG. 19D shows NO production in cyanidin 3-galactoside treated cells. The production of NO was measured by ELISA. The data are presented as mean±SE. % of control was determined from LPS treated cells without test agent treatment.

### DETAILED DESCRIPTION

Polyphenols such as chlorogenic acids, chicoric acid, quercetin derivatives, and anthocyanins have a wide range of biological and pharmacological activities. However, such polyphenols are not readily and economically available. Thus, in order to produce polyphenols in economically efficient manner, better tools for the production of polyphenols such as chlorogenic acids, chicoric acid, quercetin derivatives, and anthocyanins are needed.

Presented herein are systems and methods for increased production of polyphenols in red lettuce. The systems and methods presented herein allow for the high yield production of polyphenols for high-quantity, low-cost, scalable production of polyphenols. In particular, the systems and methods allow for production of polyphenols, such as chlorogenic acids, chicoric acid, quercetin derivatives, and anthocyanins as well as the exploration of their benefits at meaningful scale. Additionally, the systems and methods provide cost-effective production of chlorogenic acids, chicoric acid, and quercetin derivatives at commercially relevant quantities. The systems and methods presented herein utilize readily available lettuce chassis, by utilizing the naturally abundant intermediates (endogenous genes and enzymes) of the polyphenol biosynthesis pathways in lettuce with the power of metabolic engineering technologies.

The present disclosure provides red lettuces with significantly increased amounts of health beneficial polyphenols such as quercetin derivatives, chicoric acid, chlorogenic acids, and anthocyanins. Also provided herein are methods of producing such red lettuces, for example, by using eustressors/elicitors to stimulate the production of desired secondary metabolites as well as regulating genes of the phenylpropanoid pathway to enhance the downstream secondary metabolites. The disclosure also provides extracts from such lettuces, methods of making such extracts, and methods of using such extracts, for example, to inhibit viral replication, reduce inflammation, improve visual acuity, modulate the immune response, reduce obesity and diabetes, reduce blood glucose levels, or combinations thereof.

The present disclosure includes a variety of aspects, which may be combined in different ways. The following descriptions are provided to list elements and describe some of the embodiments of the present disclosure. These elements are listed with initial embodiments; however, it should be understood that these embodiments may be combined in any manner and in any number to create additional embodiments. The variously described examples and preferred embodiments should not be construed to limit the present disclosure to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

Polyphenols are beneficial plant compounds with antioxidant properties that may help keep one healthy and protect against various diseases. More than 8,000 types of polyphenols have been identified (Tsao, R. Nutrients 2010, 2(12), 1231-1246; and Zhou et al., Nutrients 2016, 8, 515). Polyphenols can be further categorized into at least four main groups, which include flavonoids, phenolic acids, polyphenolic amides, and other polyphenols. Flavonoids account for around 60% of all polyphenols. Examples include quercetin, kaempferol, catechins, and anthocyanins, which are found in foods like apples, onions, dark chocolate, and red cabbage. Phenolic acids account for around 30% of all polyphenols. Examples include stilbenes and lignans, which are mostly found in fruits, vegetables, whole grains, and seeds Polyphenolic amides include capsaicinoids in chili peppers and avenanthramides in oats. Other polyphenols include resveratrol in red wine, ellagic acid in berries, curcumin in turmeric, and lignans such as those found in flax seeds, sesame seeds, and whole grains.

Plant phenolics including simple phenols, phenolic acids, flavonoids, coumarins, stilbenes, hydrolysable and condensed tannins, lignans, and lignins are the most abundant secondary metabolites, produced mainly through the shikimate pathway from L-phenylalanine and L-tyrosine, and containing one or more hydroxyl groups attached directly to aromatic ring (Chirinos et al., Food Chem. 113 (2009) 1243-1251; and Kumar et al., Biotechnol. Rep. 4 (2014) 86-93). Secondary metabolites originate from primary metabolites (carbohydrates, amino acids, and lipids) principally for protection against UV radiation, competitive warfare against viruses, bacteria, insects and other plants, as well as responsible for smell, color and flavor in plant products (Winkel-Shirley, B. Plant Physiology. 2001,126 (2): 485-93). Plant phenolics are similar in many ways to alcohols with aliphatic structure but the presence of aromatic ring, hydrogen atom of phenolic hydroxyl group makes them as weak acids. Plant phenolics are known to exhibits a variety of functions including plant growth, development, and defense and also have beneficial effects on mankind. Plant phenolics are acknowledged as strong natural antioxidants having key role in wide range of biological and pharmacological properties such as anti-inflammatory, anticancer, antimicrobial, anti-allergic, antiviral, antithrombotic, hepatoprotective, food additive, signaling molecules and many more (Kumara et al., Biotechnol. Rep. 24 (2019) 1-10).

### Flavonoids

Flavonoids (or bioflavonoids) (from the Latin word flavus, meaning yellow, their color in nature) are a class of plant and fungus secondary metabolites (Formica et al., Food and Chemical Toxicology. 1995,33 (12): 1061-80). Flavonoids are widely distributed in plants with multiple functions. Flavonoids are the most important plant pigments for flower coloration, producing yellow or red/blue pigmentation in petals, attracting pollinating insects. Flavonoids cover a wide range of functions in higher plants such as UV filtration, symbiotic nitrogen fixation and floral pigmentation. Additionally, Flavonoids may function as chemical messengers, physiological regulators, and cell cycle inhibitors. Furthermore, some flavonoids have inhibitory activity against organisms that cause plant diseases.

The biosynthesis pathways of naturally occurring quercetin and its derivatives have been elucidated (Winkel-Shirley, B. Plant Physiology. 2001, 126 (2): 485-93). Biosynthetically, in plants, phenylalanine is converted to 4-coumaroyl-CoA in a series of steps known as the general phenylpropanoid pathway using phenylalanine ammonia-lyase (PAL), cinnamate 4-hydroxylase (C4H), and 4-coumaroyl CoA-ligase (4CL). One molecule of 4-coumaroyl-CoA is added to three molecules of malonyl-CoA to form tetrahydroxychalcone using 7,2′-dihydroxy-4′-methoxyisoflavanol synthase. Tetrahydroxychalcone is then converted into naringenin using chalcone isomerase (CHI). Naringenin is converted into eriodictyol using flavanoid 3′-hydroxylase. Eriodictyol is then converted into dihydroquercetin with flavanone 3-hydroxylase (F3H), which is then converted into quercetin using flavonol synthase (FLS). The following enzymatic glycosylation and esterification processes will generate quercetin-3-O-glucoside (Q3G) and quercetin-3-O-malonylglucoisde (Q3MG), respectively.

### Quercetin and Quercetin Derivatives

Quercetin is one of the most abundant dietary flavonoids. Quercetin can be found in many plants and foods, such as red wine, onions, green tea, apples, berries, Ginkgo biloba, St. John's wort, American elder, and others (Flavonoids, Micronutrient Information Center, Linus Pauling Institute, Oregon State University, 2015). Quercetin has been linked to improved exercise performance and reduced inflammation, blood pressure and blood sugar levels. It may also have brain-protective, anti-allergy, and anticancer, antibacterial and antiviral properties. However, quercetin is generally not sufficiently bioavailable and largely are transformed to different metabolites. Although little is known about their biological activities, these metabolites linked to the health benefits associated with quercetin dietary intake (Lesjak, M. et al. 2018 Journal of Functional Foods, 40, 68-75). Activities of quercetin and its derivatives found in plant extracts are believed to act as potent antioxidant and anti-inflammatory agents and may contribute to overall biological activity of quercetin-rich diet (Carullo, G. et al. 2017 Future medicinal chemistry, 9(1), 79-93). Quercetin derivatives include quercetin-3-O-glucuronide (Q3G) (also known as isoquercetin), tamarixetin, isorhamnetin, isorhamnetin-3-O-glucoside, quercetin-3,4′-di-O-glucoside, quercetin-3,5,7,3′,4′-pentamethyl ether. Some examples of the naturally occurring quercetin and its derivatives include quercetin-3-O-malonylglucoside (Q3MG) and quercetin-3-O-glucoside (Q3G).

### Anthocyanins

Anthocyanins are colored water-soluble pigments belonging to the phenolic group (Khoo et al., Food Nutr Res. 61(1), 2017). The pigments are in glycosylated forms. Anthocyanins responsible for the colors, red, purple, and blue, are in fruits and vegetables. Berries, currants, grapes, and some tropical fruits have high anthocyanins content. Red to purplish blue-colored leafy vegetables, grains, roots, and tubers are the edible vegetables that contain a high level of anthocyanins. Among the anthocyanin pigments, cyanidin-3-glucoside is the major anthocyanin found in most of the plants. Anthocyanins possess antidiabetic, anticancer, anti-inflammatory, antimicrobial, and anti-obesity effects, as well as prevention of cardiovascular diseases (He et al., J Ethnopharmacol. 137(3) (2011):1135-1142.

### Phenolic Acids

The term “phenolic acids” generally describes the phenolic compounds having one carboxylic acid group. Phenolic or phenol carboxylic acids (a type of phytochemical called a polyphenol) are one of the main classes of plant phenolic compounds. Phenolic acids are found in the variety of plant-based foods such as seeds, skins of fruits and leaves of vegetables that contain them in highest concentrations. Typically, phenolic acids are present in bound form such as amides, esters, or glycosides and rarely in free form (Pereira et al., Molecules 14 (6) (2009) 2202-2211). Phenolic acids are often divided in to two sub-groups: hydroxybenzoic acid and hydroxycinnamic acid (Clifford et al., J. Sci. Food Agric. 79 (1999) 362-372). Phenolic acids possess much higher in vitro antioxidant activity than well-known antioxidant vitamins (Tsao et al., J. Chromatogr. B Analyt. Technol. Biomed. Life Sci. 812 (2004) 85-99).

Hydroxycinnamic acids (HCAs), derived from cinnamic acid, present in foods often as simple esters with quinic acid or glucose. The most abundant soluble bound hydroxycinnamic acid present is chlorogenic acid (a combined form of caffeic and quinic acids). The four most common hydroxycinnamic acids are ferulic, caffeic, p-coumaric, and sinapic acids.

Hydroxybenzoic acids possess a common structure of C6-C1 and derived from benzoic acid. Hydroxybenzoic acids are found in soluble form (conjugated with sugars or organic acids) and bound with cell wall fractions such as lignin (Strack et al., Plant Biochemistry, Academic, London, 1997, pp. 387; and Khoddami et al., Molecules 18 (2013) 2328-2375). As compared to hydroxycinnamic acids, hydroxybenzoic acids are generally found in low concentration in red fruits, onions, black radish, etc., (Shahidi et al., Technomic Publishing Co., Inc., Lancaster, PA, 1995). The four commonly found hydroxybenzoic acids are p-hydroxybenzoic, protocatechuic, vanillic, and syringic acids.

### Chlorogenic Acid

One type of biologically active phenolic acids, chlorogenic acid (CGA) is the ester of caffeic acid and (−)-quinic acid, functioning as an intermediate in lignin biosynthesis. The term “chlorogenic acids” refers to a related polyphenol family of esters, including hydroxycinnamic acids (caffeic acid, ferulic acid, and p-coumaric acid) with quinic acid. Examples of chlorogenic acids include 5-O-caffeoylquinic acid (chlorogenic acid or 5-CQA), 4-O-caffeoylquinic acid (cryptochlorogenic acid or 4-CQA), and 3-O-caffeoylquinic acid (neochlorogenic acid or 3-CQA).

### 5-O-Caffeoylquinic Acid

Biosynthetically, the initial steps in the biosynthesis of CQAs are via the phenylpropanoid pathway and the enzymes catalyzing the conversions. The conversion of phenylalanine to p-coumaroyl-CoA, with cinnamic acid and p-coumaric acid acting as intermediates, is catalyzed sequentially by phenylalanine ammonia lyase (PAL), cinnamate 4-hydroxylase (C4H) and 4-cinnamoyl-CoA ligase (4CL).

### Chicoric Acid

Chicoric acid (also known as cichoric acid) is a hydroxycinnamic acid, an organic compound of the phenylpropanoid class and occurs in a variety of plant species. It is a derivative of both caffeic acid and tartaric acid (Shi et al., Functional Foods: Biochemical and Processing Aspects. CRC Press. 2(27) (2002) pp. 241). Chicoric acid has been shown to stimulate phagocytosis in both in vitro and in vivo studies, to inhibit the function of hyaluronidase (an enzyme that breaks down hyaluronic acid in the human body), to protect collagen from damage due to free radicals, and to inhibit the function of HIV-1 integrase.

### Definitions

“Flavonoid” refers to a diverse family of aromatic molecules that are derived from phenylalanine and malonyl-coenzyme A (CoA; via the fatty acid pathway). These compounds include six major subgroups that are found in most higher plants: chalcones, flavones, flavonols, flavandiols, anthocyanins, and condensed tannins (or proanthocyanidins); a seventh group, the aurones, is widespread, but not ubiquitous. Examples of efforts to elucidate biosynthetic pathways of flavonoid production from a genetic perspective are provided in Ferreyra, M. et al., Frontiers in Plant Science, 2012, 3, 222 and Winkel-Shirley, B. Plant Physiol. 2001,126, 485-493. Biosynthetically, flavonoids are synthesized through the phenylpropanoid pathway, transforming phenylalanine into 4-coumaroyl-CoA, which finally enters the flavonoid biosynthesis pathway. Without wishing to be bound by theory, it is thought that the first enzyme specific for the flavonoid pathway, chalcone synthase (CHS), produces chalcone scaffolds from which all flavonoids derive.

Chemically, flavonoids have the general structure of a 15-carbon skeleton, which consists of two phenyl rings (A and B) and a heterocyclic ring (C). This carbon structure can be abbreviated C6-C3-C6. The general structure of flavonoids is provided as Formula (I).

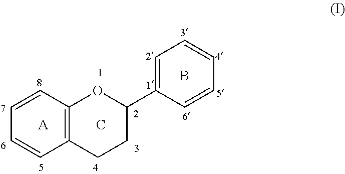

“Polyphenols” as used herein refers to organic chemicals that include more than one phenol structural units. Polyphenols commonly found in lettuce include anthocyanins, chicoric acid, chlorogenic acids, dicaffeoylquinic acids and quercetin derivatives.

As used herein “eustressor” and “elicitor” are used interchangeably and refer to various biological, physical or chemical stressful factors that trigger the signaling pathways leading to a higher bioactive compounds content and quality attributes of plant products. Eustressors/elicitors can be classified as biotic and abiotic substances, examples of which are provided in Table 1. Plant hormones/plant growth regulators (e.g., salicylic acid (SA), jasmonates, etc.) are also considered as eustressors/elicitors. Eustressors/elicitors of biological, chemical, or physical origin may increase plant agronomic/nutrition traits due to the activation of responses that could include defense responses among them, leading to an increase of functional quality of, e.g., fruits and vegetables. Plant growth regulators (PGRs) can be used as eustressors/elicitors to stimulate production of plant secondary metabolites. Plant growth regulators can include hormonal substances of natural occurrence (phytohormones) as well their synthetic analogues.

TABLE 1
Examples of eustressor/elicitor classification based on source/origin
Biotic Elicitors
Lipopolysaccharides
Polysaccharides: Pectin and cellulose (cell walls); chitosan, chitin and
glucans (microorganisms), alginate, arabic gum, guar gum, LBG, yeast
extract.
Oligosaccharides: Galacturonides, guluronate, mannan, mannuronate.
Proteins: Cellulase, cryptogein, glycoproteins, oligandrin, pectolyase,
fish protein hydrolysates, lactoferrin.
Complex composition: Fungal spores, mycelia cell wall, microbial cell
wall.
Pathogen toxin: Coronatine.
Oregano extract
Abiotic Elicitors

| | |
|---|---|
| Chemical | Physical |
| Acetic acid | Altered gas composition |
| Benzothiadiazole | Chilling |
| Silicon | CO₂ |
| Bioregulator prohexadione | Drought |
| Ethanol | Extreme temperature |
| | shock |
| Ethene | High pressure |
| Inorganic salts: mercuric chloride (HgCl₂), | High or low osmolarity, |
| copper sulfate (CuSO₄), calcium chloride | UV irradiation |
| (CaCl₂), and vanadyl sulfate. | |
| Saline stress Metal ions: Co²⁺, Fe²⁺, Al³⁺, | Wounding, Ozone |
| Ag²⁺, Ag⁺, Mn²⁺, Zn²⁺, Cu²⁺, Pb²⁺ | |
| and Cd²⁺ | |

Plant Growth Regulators
Plant growth regulators include hormonal substances of natural
occurrence (phytohormones) as well as their synthetic analogues

“Plant” includes the whole plant or any parts such as plant organs (e.g., harvested or non-harvested leaves, etc.), plant cells, plant protoplasts, plant cell or tissue cultures from which whole plants can be regenerated, plant callus, plant cell clumps, plant transplants, seedlings, plant cells that are intact in plants, plant clones or micropropagations, or parts of plants (e.g., harvested tissues or organs), such as plant cuttings, vegetative propagations, embryos, pollen, ovules, flowers, leaves, heads, seeds, clonally propagated plants, roots, stems, stalks, root tips, grafts, parts of any of these and the like, or derivatives thereof, preferably having the same genetic make-up (or very similar genetic make-up) as the plant from which it is obtained. In addition, any developmental stage is included, such as seedlings, cuttings prior or after rooting, mature and/or immature plants or mature and/or immature leaves.

“Lettuce” refers herein to plants of the species Lactuca sativa L. Lactuca sativa is in the Cichorieae tribe of the Asteraceae (Compositae) family. Lettuce is related to chicory, sunflower, aster, dandelion, artichoke, and chrysanthemum. L. sativa is one of about 300 species in the genus Lactuca. As a highly polymorphic species, L. sativa is grown for its edible head and leaves. As a crop, lettuce is grown commercially anywhere environmental conditions permit the production of an economically viable yield. Fresh lettuce is consumed nearly exclusively as fresh, raw product and occasionally as a cooked vegetable. Lettuce is an increasingly popular crop. Lettuce consumption continues to increase worldwide. Due to its high demands, there are benefits to seeking increased in production of polyphenols for new transgenic lettuces. In particular, improved transgenic lettuce with enhanced production of health polyphenols that are stable, high yielding, and agronomically sustainable will particularly be commercially viable for human consumption.

“Lettuce plant” refers to an immature or mature lettuce plant, including a whole lettuce plant and a lettuce plant from which seed, roots or leaves have been removed. A seed or embryo that will produce the plant is also considered to be the lettuce plant. Lettuce plants can be produced by seeding directly in the ground (e.g., soil such as soil on a field) or by germinating the seeds in a controlled environment condition (e.g., a greenhouse) and then transplanting the seedlings into the field. See, e.g., Gonai et al., J. of Exp. Bot., 55(394), 111-118, 2004; Louise Jackson et al, Acquaah, Principles of Plant Genetics and Breeding, 2007, Blackwell Publishing, and Jackson, Louise, et al, University of California, Publication 7216 which are all herewith incorporated by reference.

“Lettuce cell” or “lettuce plant cell” refers to a lettuce cell that has been isolated, is grown in tissue culture, and/or is incorporated in a lettuce plant or lettuce plant part.

“Lettuce plant parts” as used herein includes lettuce heads, lettuce leaves, parts of lettuce leaves, pollen, ovules, flowers, and the like. In another embodiment, the present disclosure is further directed to lettuce heads, lettuce leaves, parts of lettuce leaves, flowers, pollen, and ovules isolated from lettuce plants.

The term “variety” or “cultivar” means a plant grouping within a single botanical taxon of the lowest known rank, which grouping, irrespective of whether the conditions for the grant of a breeder's right are fully met, can be defined by the expression of the characteristics resulting from a given genotype or combination of genotypes, distinguished from any other plant grouping by the expression of at least one of the said characteristics and considered as a unit with regard to its suitability for being propagated unchanged.

As used herein, a polynucleotide or polypeptide is “recombinant” when it is artificial or engineered, or derived from an artificial or engineered protein or nucleic acid. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A polypeptide expressed in vitro or in vivo from a recombinant polynucleotide is an example of a recombinant polypeptide. Likewise, a polynucleotide sequence that does not appear in nature, for example, a variant of a naturally occurring gene is recombinant.

As used herein, “heterologous” in reference to a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

“Transgene” as used herein refers to a gene or genetic transferred into the genome of a lettuce plant, for example by genetic engineering methods, such as by transformation. Exemplary transgenes include cDNA (complementary DNA) segment, which is a copy of mRNA (messenger RNA), and the gene itself residing in its original region of genomic DNA. In one example, describes a segment of DNA containing a gene sequence that is introduced into the genome of a lettuce plant or lettuce plant cell. This non-native segment of DNA may retain the ability to produce RNA or protein in the transgenic lettuce plant, or it may alter the normal function of the transgenic plant's genetic code. In general, the transferred nucleic acid is incorporated into the plant's germ line. Transgene can also describe any DNA sequence, regardless of whether it contains a gene coding sequence or it has been artificially constructed, which has been introduced into a lettuce plant or vector construct in which it was previously not found.

“Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or noncontiguous. When used to refer to the joining of two protein coding regions, by operably linked is intended that the coding regions are in the same reading frame. The cassette may additionally contain at least one additional coding sequence/gene to be co-transformed into the organism. Alternatively, the additional coding sequences/gene(s) can be provided on multiple expression cassettes. Such an expression cassette is provided with a plurality of restriction sites and/or recombination sites for insertion of a coding polynucleotide of interest or active variant or fragment thereof to be under the transcriptional regulation of the regulatory regions (e.g., promoter). The expression cassette may additionally contain selectable marker genes.

“Expression cassette” refers a polynucleotide encoding a polypeptide of interest operably linked to at least one polynucleotide encoding an expression control sequence. The expression cassette can include in the 5′-3′ direction of transcription, a transcriptional and translational initiation region (i.e., a promoter), polynucleotide encoding a polypeptide of interest or active variant or fragment thereof, and a transcriptional and translational termination region (i.e., termination region) functional in plants. The regulatory regions (i.e., promoters, transcriptional regulatory regions, and translational termination regions) and/or the polynucleotide or active variant or fragment thereof may be native/analogous to the host cell or to each other. Alternatively, the regulatory regions and/or the polynucleotide of or active variant or fragment thereof may be heterologous to the host cell or to each other.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385. See also Della-Cioppa et al. (1987) Plant Physiol. 84:965-968.

“Expression control sequence” refers to a segment of a nucleic acid molecule which is capable of increasing or decreasing the expression of a polypeptide encoded by the expression cassette. Examples of expression control regions include promoters, transcriptional regulatory regions, and translational termination regions. The termination region may be native with the transcriptional initiation region, may be native with the operably linked polynucleotide or active variant or fragment thereof, may be native with the plant host, or may be derived from another source (i.e., foreign or heterologous) to the promoter, the polynucleotide or active fragment or variant thereof, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262: 141-144; Proudfoot (1991) Cell 64:671-674; Sanfacon et al. (1991) Genes Dev. 5: 141-149; Mogen et al. (1990) Plant Cell 2: 1261-1272; Munroe et al. (1990) Gene 91: 151-158; Ballas et al. (1989) Nucleic Acids Res. 17:7891-7903; and Joshi et al. (1987) Nucleic Acids Res. 15:9627-9639.

“Variant” protein is intended to mean a protein derived from the protein by deletion (i.e., truncation at the 5′ and/or 3′ end) and/or a deletion or addition of one or more amino acids at one or more internal sites in the native protein and/or substitution of one or more amino acids at one or more sites in the native protein. Variant proteins encompassed are biologically active, that is they continue to possess the desired biological activity of the native protein.

A “plant bio-stimulant” as used herein, refers to a material which contains substance(s) and/or microorganisms that, when applied to plants or the rhizosphere, stimulates natural processes to enhance and/or improve nutrient uptake, nutrient efficiency, tolerance to abiotic stress, and crop quality, independent of its nutrient content. In some embodiments, a bio-stimulant is a biotic eustressor/elicitor.

A “control” or “control lettuce” or “control lettuce cell” provides a reference point for measuring changes in phenotype of the subject lettuce plant or lettuce plant cell, and may be any suitable lettuce plant or lettuce cell. A control lettuce or lettuce cell may comprise, for example: (a) a wild-type or native lettuce or lettuce cell, i.e., of the same genotype as the starting material for the genetic alteration which resulted in the subject lettuce or lettuce cell; (b) a lettuce or lettuce cell of the same genotype as the starting material but which has been transformed with a null construct (i.e., with a construct which has no known effect on the trait of interest, such as a construct comprising a marker gene); (c) a lettuce or lettuce cell which is a non-transformed segregant among progeny of a subject lettuce or lettuce cell; (d) a lettuce or lettuce cell which is genetically identical to the lettuce or lettuce cell but which is not exposed to the same treatment (e.g., eustressor/elicitor treatment, herbicide treatment) as the subject lettuce or lettuce cell; or (e) the subject lettuce or lettuce cell itself, under conditions in which the gene of interest is not expressed.

An “effective amount” or a “therapeutically effective amount” may refer to an amount of therapeutic agent (e.g., a lettuce extract, lettuce plant, or lettuce plant part described herein) that provides a desired physiological change, such as an anti-viral, anti-inflammatory, anti-oxidant, and/or anti-cancer effect). The desired physiological change may be, for example, a decrease in symptoms of a disease, or a decrease in severity of a disease, or may be a reduction in the progression of a disease. With respect to viral infection, the desired physiological changes may include, for example, decreased detectable virus in a subject, decreased symptoms, decreased viral replication, and/or decreased virus binding to host cells. With respect to cancer, the desired physiological changes may include, for example, tumor regression, a decreased rate of tumor progression, a reduced level of a cancer biomarker, reduced symptoms associated with cancer, a prevention or delay in metastasis, or clinical remission.

In the present description, the term “about” means ±20% of the indicated range, value, or structure, unless otherwise indicated. The term “consisting essentially of” limits the scope of a claim to the specified materials or steps and those that do not materially affect the basic and novel characteristics of the claimed embodiment. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof.

Recombinant DNA, molecular cloning, and gene expression techniques used in the present disclosure are known in the art and described in references, such as Sambrook et al., Molecular Cloning: A Laboratory Manual, 3^{rd} Ed., Cold Spring Harbor Laboratory, New York, 2001, and Ausubel et al., Current Protocols in Molecular Biology, John Wiley and Sons, Baltimore, MD, 1999.

All documents (e.g., patent publications) are herein incorporated by reference in their entirety.

Various modifications and variations of the described products and methods of the present disclosure will be apparent to those skilled in the art without departing from the scope and spirit of the present disclosure. Although the present disclosure has been described in connection with specific embodiments, it should be understood that the present disclosure as claimed should not be unduly limited to such specific embodiments.

### Improved/Increased Polyphenol Production in Plant Systems

As discussed above, polyphenols such as flavonoids, anthocyanins, chicoric acid, and chlorogenic acids share a common biosynthetic phenylpropanoid pathway. Accordingly, provided herein are strategies to regulate their production in a plant system.

Coupling regulation of general genes with specific genes for the targeted polyphenols can be used to produce specific polyphenols in an efficient and economical way. Obtaining highly bioavailable quercetin derivatives (more water-soluble) is the advantageous to producing biologically effective products. Since the endogenous biosynthetic pathways to quercetin and derivatives already exist in a red lettuce system, one objective of the instant disclosure to construct a target-directed and more efficient bio-engineered system. The main strategy of the present disclosure is to utilize readily available plant chassis, by coupling the naturally abundant flavonoid intermediates, endogenous genes, and enzymes in plants with the power of synthetic biology technologies. Moreover, lettuce is a high bio-mass, fast growing, and very popular vegetable.

The phytochemical composition of plants as foods varies with genetics (family, species, cultivar, etc.), physiological (organ, maturity and age) and agronomical factors (photoperiod, chemical stressors, etc.) (Nieves B. et al., Molecules 2014, 19, 13541-13563.; Bellostas, N. et al., Sci. Hortic. 2007, 114, 234-242; Cartea, M. E. et al., Phytochem. Rev. 2008, 7, 213-229; Charron, C. S. et al., J. Sci. Food Agric. 2005, 85, 671-681; Dominguez-Perles, R. et. al., J. Food Sci. 2010, 75, C383—C392; Francisco, M., et al., J. Chromatogr. A 2009, 1216, 6611-6619; Pérez-Balibrea, S. J. Clin. Biochem. Nutr. 2008, 43, 1-5; and Pérez-Balibrea, S., et al., J. Sci. Food Agric. 2008, 88, 904-910). These factors are categorized as biotic (genetics, physiological determinants, pests and diseases) and abiotic (environment and agronomical conditions) and may be used to enhance valuable metabolites in foods and ingredients, in a year-round production. Specific treatments, including the eustressor/elicitor application can be used to increase metabolite production in the plant and to enhance its qualitative value for fresh produce, enriched food, or as a raw ingredient for feed/food and pharmaceutical products.

The present disclosure includes systems for biosynthesis of polyphenols in lettuce. “System for biosynthesis of polyphenols in lettuce” refers to a system that when introduced into a red lettuce allows for increased production of polyphenols when the system is applied to a lettuce. In some embodiments, the systems include at least one eustressor/elicitor, or a homologue, isomer or derivative thereof, that increase the production of polyphenols in lettuce. In some embodiments, the systems include an expression cassette comprising a heterologous expression control sequence operably linked to at least one polynucleotide encoding one or more proteins that increase the production of polyphenols in lettuce. In some embodiments, the systems include the at least one eustressor/elicitor, or a homologue, isomer or derivative thereof of the present disclosure; and the expression cassette of the present disclosure. In some embodiments, the system is for use in a method for biosynthesis of polyphenols in lettuce, the method comprising administering at least one eustressor/elicitor, or a homologue, isomer or derivative thereof, to the lettuce, thereby increasing the production of polyphenols in lettuce.

In some embodiments, the system for biosynthesis of polyphenols in lettuce comprise at least one eustressor/elicitor, or a homologue, isomer or derivative thereof, that increase the production of polyphenols in lettuce. In some embodiments, provided herein is a method for biosynthesis of polyphenols in lettuce, comprising administering at least one eustressor/elicitor, or a homologue, isomer or derivative thereof, to the lettuce, thereby increasing the production of polyphenols in lettuce. In some embodiments, combinations i.e., one or more, of eustressor/elicitors have been used for the high production of desired health beneficial polyphenols in applied red lettuces. Without wishing to be bound by a particular theory, the increase in phytochemicals could be linked by the increase of gene transcripts of genes involved in pathways that result in biosynthesis of polyphenols, which leads to an enhanced phytochemical biosynthesis. In some embodiments, significant enhancement of health beneficial polyphenol contents in red lettuces has been achieved by combinations, i.e., one or more, eustressor/elicitors.

In some embodiments, the at least one eustressor/elicitor is a plant growth regulator. In some embodiments, the plant growth regulator is selected from: auxins, cytokinins (CKs), gibberellins (GAs), ethylene, brassinosteroids, jasmonates (JAs), strigolactones (SLs), salicylic acid (SA), and any homologues or isomers or derivatives, synthetic analogues, or any combination or mixture thereof. In some embodiments, the plant growth regulator is phytohormones.

In some embodiments, the at least one eustressor/elicitor is selected from: arachidonic acid (AA), indole-3-acetic acid (IAA), 5-aminolevumic acid (5-ALA), harpin protein (HP), or any combination or mixture thereof.

In some embodiments, the at least one eustressor/elicitor is selected from: indole-3-acetic acid (IAA), indole-3-acetonitril (IAN), indole-3-acetaldehyde (IAc), ethylindoeacetate, indole-3-pyruvic acid (IPyA), indole-3-butyric acid (IBA), indole-3-propionic acid (IPA), indazole-3-acetic acid, chlorophenoxypropionic acids, naphthalene acetic acid (NAA), phenoxy acetic acid (PAA), 2,4-dichlorophenoxy acetic acid (2,4-D), 2,4,5-trichlorophenoxy acetic acid (2,4,5-T), naphthalene acetamide (NAAM), 2-napthoxyacetic acid (NOA), 2,3,5-triodobenzoic acid (TIBA), thianaphthen-3-propionic acid (IPA), ribosylzeatin, zeatin, isopentinyladenine, dihydrozeatin, 6-benzyl amino purine, 6-phenyl amino purine, kinetin, N-benzyl-9-(2-tetrahydropyranyl) adenine (BPA), diphenylurea, thidiazuron, benzimidazole, adenine, 6-(2-thenylamino) purine, GA, GA4, GA7, GA3, ethylene, ethephon, ethrel, dolicholide, 28-homodolicholide, castasterone, dolichosterone, 28-homodolichosterone, typhasterol, jasmonic acid, methyl dihydrojasmonate, dihydrojasmonic acid, methyl jasmonate (MJ), strigol, orobanchol, GR24, arachidonic acid (AA), salicylic acid (SA), Harpin protein (HP), or any combination or mixture thereof.

In some embodiments, the at least one eustressor/elicitor is selected from: indole-3-acetic acid (IAA), naphthalene acetic acid (NAA), oxalic acid, benzothiadiazole (BTH), 2,4-dichlorophenoxy acetic acid (2,4-D), arachidonic acid (AA), salicylic acid (SA), methyl jasmonate (MJ), harpin protein (HP), or any ombination or mixture thereof.

In some embodiments, the at least one eustressor/elicitor is selected from: lipopolysaccharides, pectin and cellulose (cell walls); chitosan, chitin and glucans (microorganisms), alginate, arabic gum, guar gum, LBG, yeast extract, galacturonides, guluronate, mannan, mannuronate, cellulase, cryptogein, glycoproteins, oligandrin, pectolyase, fish protein hydrolysates, lactoferrin, fungal spores, mycelia cell wall, microbial cell wall, coronatine, cregano extract, reynoutria sachalinensis extract; or any combination or mixture thereof.

In some embodiments, the at least one eustressor/elicitor is selected from the following plant bio-stimulant categories: humic and fulvic acids; protein hydrolysates and other N-containing compounds; seaweed extracts and botanicals; chitosan and other biopolymers; inorganic compounds; beneficial fungi; beneficial bacterial; or any combination or mixture thereof.

In some embodiments, the system comprises the eustressor/elicitor at a concentration of about 30 mg/L to 1000 mg/L. In some embodiments, the system comprises the eustressor/elicitor at a concentration of about 30 mg/L to 500 mg/L, 30 mg/L to 400 mg/L, 30 mg/L to 300 mg/L, 30 mg/L to 200 mg/L, 30 mg/L to 150 mg/L, 30 mg/L to 100 mg/L. In some embodiments, the system comprises the eustressor/elicitor at a concentration of about 30 mg/L, 60 mg/L, 120 mg/L, or 200 mg/L.

In some embodiments, the system comprises the eustressor/elicitor at a concentration of about 1 μM to 1000 μM. In some embodiments, the system comprises the eustressor/elicitor at a concentration of about 1 μM to 900 μM, 1 μM to 800 μM, 1 μM to 700 μM, 1 μM to 600 μM, 1 μM to 500 μM, 1 μM to 400 μM, 1 μM to 300 μM, 1 μM to 200 μM, 1 μM to 100 μM, 5 μM to 100 μM, or 5 μM to 90 μM. In some embodiments, the system comprises the eustressor/elicitor at a concentration of about 5 μM, 10 μM, 15 μM, 45 μM, or 90 μM.

In some embodiments, the system comprises the eustressor/elicitor selected from: indole-3-acetic acid (IAA), naphthalene acetic acid (NAA), 2,4-dichlorophenoxy acetic acid (2,4-D), arachidonic acid (AA), salicylic acid (SA), and/or methyl jasmonate (MJ), wherein each eustressor/elicitor is independently at a concentration of about 1 μM to 100 μM. In some embodiments, each eustressor/elicitor is independently at a concentration of about 5 μM, 10 μM, 15 μM, 45 μM, or 90 μM.

In some embodiments, the system comprises the eustressors/elicitors harpin protein (HP), chitosan, alginate, arabic gum, guar gum, and/or yeast extract, at a concentration in a range of about 30-200 mg/L. In some embodiments, the system comprises a eustressors/elicitors comprising at least one of plant-based extract at a concentration in a range of about 100-5000 mg/L. In some embodiments, the system comprises the eustressors/elicitors harpin protein (HP), chitosan, alginate, arabic gum, guar gum, yeast extract, at a concentration of about 30 mg/L, 60 mg/L, 120 mg/L, or 200 mg/L.

In some embodiments, the polyphenol of the present disclosure, is chlorogenic acid or derivatives thereof, chicoric acid, and/or water-soluble quercetin derivative. In some embodiments, the chlorogenic acid is 3-O-caffeoylquinic acid (3-CQA), 4-O-caffeoylquinic acid (4-CQA), and/or 5-O-caffeoylquinic acid (5-CQA); the chicoric acid is (2R,3R)-O-dicaffeoyltartaric acid; and/or wherein the water-soluble quercetin derivative is quercetin-3-O-glucoside (Q3G) and/or quercetin-3-O-malonylglucoside (Q3MG). In some embodiments, the increased production of polyphenols is quantified by LC-MS. In some embodiments, the increased production of polyphenols is quantified by HPLC.

In some embodiments, the increased production of polyphenols is a 3- to 9-fold increased production, compared to a control system. In some embodiments, a combination of eustressors/elicitors results in an additive or synergistic effect resulting in increased production of polyphenols. In some embodiments, the control system is a system without the at least one eustressor/elicitor, or a homologue, isomer or derivative thereof.

In certain embodiments, the present disclosure relates to novel systems, methods and compositions for the in vivo/in vitro production, modification and isolation of flavonoids, chlorogenic acids, chicoric acid, and anthocyanins compounds from plant or enzymatic systems, including whole lettuce plants, lettuce plant parts, and/or lettuce plant cell suspension cultures systems or enzymatic bioconversion systems. In certain embodiments, the present disclosure provides a novel system of genetically modifying a lettuce plant or plant cell suspension culture to produce, modify and/or accumulate health beneficial polyphenols in red lettuces.

In some embodiments, the system for biosynthesis of polyphenols in lettuce comprises an expression cassette comprising a heterologous expression control sequence operably linked to at least one polynucleotide encoding one or more proteins that increase the production of polyphenols in lettuce.

In some embodiments, the one or more proteins comprise malonate-CoA ligase. In such embodiments, the system includes one or more polynucleotide encoding a malonate-CoA ligase. Malonate-CoA ligase catalyzes the formation of malonyl-CoA, which is a precursor of flavonoid biosynthesis, directly from malonate and CoA. The malonate-CoA ligase may be AAE13. In some embodiments, the malonate-CoA ligase is AAE13. Some examples of transgenes used for engineering biosynthesis of malonyl-CoA and boosting building blocks for the health beneficial polyphenol synthesis are AAE13 (malonate-CoA ligase) and AtMYB12 transcription factor.

In some embodiments, the system includes one or more polynucleotides encoding an enzyme of the phenylpropanoid pathway. In particular embodiments, the enzymes of the phenylpropanoid pathway are selected from: phenylalanine ammonia-lyase (PAL), cinnamic acid 4-hydroxylase (C4H), and 4-coumaric acid: CoA ligase (4CL), or any combination thereof.

In some embodiments, the system includes one or more polynucleotides encoding an enzyme of the chlorogenic acid pathway. In particular embodiments, the enzymes of the chlorogenic acid pathway are selected from: hydroxycinnamoyl CoA: quinate hydroxycinnamoyl transferase (HQT), p-coumaroyl-3-hydroxylase (C3H), and caffeoyl-CoA-3-O-methyltransferase (CCoAMT), or any combination thereof.

In some embodiments, the system includes one or more polynucleotides encoding an enzyme of the flavonoid pathway. In particular embodiments, the enzymes of the flavonoid pathway are selected from: chalcone synthase (CHS), chalcone isomerase (CHI), flavanone 3-hydroxylase (F3H), and flavonol synthase (FLS), flavonoid 3′-hydroxylase (F3′H), p-coumarate 3-hydroxylase (C3H), cinnamate 4-hydroxilase (C4H), 4-hydroxycinnamoyl-CoA ligase (4CL), hydroxycinnamoyl-CoA shikimate/quinate hydroxycinnamoyl transferase (HCT), hydroxycinnamoyl-CoA quinate hydroxycinnamoyl transferase (HQT), or any combination thereof.

In certain embodiments, the system includes one or more polynucleotides encoding a cytochrome P450 3A4, CYP oxidoreductase, and UDP-glucuronosyltransferase, or any combination thereof. P450 3A4, CYP oxidoreductase, and UDP-glucuronosyltransferase, are enzymes that may be used for producing a flavonoid gluconuride. A glucuronide, also known as glucuronoside, is any substance produced by linking glucuronic acid to another substance via a glycosidic bond. The gluconuride modification is useful, for example, for improving the water solubility of a flavonoid.

In some embodiments, system includes one or more polynucleotides encoding a transcription factor. The transcription factor may enhance production of one or more flavonoid precursors or intermediates. In certain embodiments, the present disclosure generates a genetically modified or transgenic plant that overexpresses one or more transcription factors, such as MYB transcription factors, that enhance metabolite flux through the flavonoids and chlorogenic acid, and anthocyanin biosynthetic pathways. In some embodiments, polynucleotides encode a MYB transcription factor. In certain embodiments, these transcription factors may include various analogues. In certain embodiments, one or more of the transgenes may be operably-linked to one or more promoters that are regulated by the transcription factors.

In some embodiments, the MYB transcription factor is selected from:

ELONGATED HYPOCOTYL 5 (HY5), AtCPC, AtMYBL2, AtMYB11, AtMYB12, AtMYB60, AtMYB75/PAP1, AtMYB90/PAP2, AtMYB111, AtMYB113, AtMYB114, AtMYB123/TT2, HvMYB10, BoMYB2, PURPLE (PR), MrMYB1 SmMYB39, GMYB10, V1MYBA1-1, V1MYBA1-2, V1MYBA1-3, V1MYBA2, VvMYBA1, VvMYBA2, VvMYBC2-L1, VvMYBF1, VvMYBPA1, VvMYBPA2, VvMYB5a, VvMYB5b, EsMYBA1, GtMYBP3, GtMYBP4, InMYB1, BoPAP1, MYB110a, DkMYB2, DkMYB4, LEGUME ANTHOCYANIN PRODUCTION1 (LAP1), MtPAR, LhMYB6, LhMYB12, LhMYB12-Lat, LjMYB14, LjTT2a, LjTT2b, LjTT2c, ZmC1, ZmPL, ZmPL-BLOTCHED1 (PL-BH), ZmP1, ZmMYB-IF35, GmMYB10, PpMYB10, PpMYBPA1, CsRUBY, OgMYB1, PcMYB10, PyMYB10, Petunia AN2, Petunia DPL, Petunia PHZ, PhMYBx, PhMYB27, PtMYB134, PtoMYB216, StAN1, StAN2, StMTF1, TaMYB14, AmROSEA1, AmROSEA2, VENOSA, SorghumY1, GmMYB176, GmMYB-G20-1, GmMYB12B2, FaMYB1, FaMYB9, FaMYB10, FaMYB11, PvMYB4a, NtAN2, LeANT1, S1MYB12, S1MYB72 AmDEL, FaMYB10, FavbHLH, and cannabis MYB12-like, and analogues thereof. In some embodiments, the MYB transcription factor is AtMYB12.

In some embodiments, the system of the present disclosure produces polyphenols that are chlorogenic acids or water-soluble quercetin derivatives. In certain embodiments, the chlorogenic acid is 3-O-caffeoylquinic acid (3-CQA), 4-O-caffeoylquinic acid (4-CQA), and/or 5-O-caffeoylquinic acid (5-CQA). In certain embodiments, the water-soluble quercetin derivative is quercetin-3-O-glucoside (Q3G) and/or quercetin-3-O-malonylglucoside (Q3MG). In some embodiments, the increased production of polyphenol is quantified by LC-MS. In some embodiments, the increased production of polyphenol is quantified by HPLC. In some embodiments, the increased production of polyphenols is a 2- to 5-fold increased production, compared to a control system. In some embodiments, the control system is a system without the expression cassette.

For any of the polynucleotides of the system, the polynucleotide may be codon-optimized for expression in a lettuce cell. In particular embodiments, the polynucleotide may be codon-optimized for expression in a red lettuce cell.

In some embodiments, the heterologous expression control sequence comprises a promoter that is functional in a plant cell. In some embodiments, the promoter is a constitutively active plant promoter. In some embodiments, the promoter is a tissue-specific promoter. In particular embodiments, the tissue-specific promoter is a leaf specific promoter. In some embodiments, the promoter is an inducible promoter. In some embodiments, the polynucleotide further comprises a regulator sequence selected from: 5′ UTRs located between a promoter sequence and a coding sequence that function as a translation leader sequence, 3′ non-translated sequences, 3′ transcription termination regions, and polyadenylation regions. A number of promoters have utility for plant gene expression for any gene of interest including but not limited to selectable markers, genes for pest tolerance, disease resistance, nutritional enhancements, and other genes of agronomic interests.

Some examples of constitutive promoters useful for lettuce plant gene expression include, but are not limited to the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2: 163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-specific promoters can be utilized to target enhanced expression within a particular plant tissue. Tissue-preferred promoters include those described in

Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2): 157-168; Rinehart et al. (1996) Plant Physiol. 112(3): 1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20: 181-196; Orozco et al. (1993) Plant Mol Biol. 23(6): 1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-specific promoters are known in the art. See, e.g., Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6): 1129-1138; and Matsuoka et al. (1993) Proc. Natl. Acad. Sci. USA 90(20):9586-9590.

Synthetic promoters are also known in the art. Synthetic constitutive promoters are disclosed in, for example, U.S. Pat. Nos. 6,072,050 and 6,555,673.

In some embodiments, the system for increasing production of polyphenols in lettuce comprise: the at least one eustressor/elicitor, or a homologue, isomer or derivative thereof of the present disclosure; and the expression cassette of the present disclosure.

For any of the polynucleotides of the system, the polynucleotide may be included in a plant transformation vector. “Transformation” refers to the introduction of new genetic material (e.g., exogenous transgenes or in the form of an expression cassette) into lettuce plant cells lettuce plant. Exemplary mechanisms that are to transfer DNA into lettuce plant cells include (but not limited to) electroporation, microprojectile bombardment, Agrobacterium-mediated transformation and direct DNA uptake by protoplasts. Transformation of plant protoplasts can also be achieved using methods based on calcium phosphate precipitation, polyethylene glycol treatment, electroporation, and combinations of these treatments (see, e.g., Potrykus et al., 1985; Omirulleh et al., 1993; Fromm et al., 1986; Uchimiya et al., 1986; Marcotte et al., 1988). Transformation of plants and expression of foreign genetic elements is exemplified in Choi et al. (1994) and Ellul et al. (2003).

“Plant transformation vector” as used herein refers to a DNA molecule used as a vehicle of delivery foreign genetic material into a plant cell. An expression cassette may be a component of a vector (e.g., a plant transformation vector), and multiple expression cassettes may be present together in a single vector. For example, a vector may encode multiple proteins of interest (e.g., two different flavonoid biosynthesis enzymes, or a single flavonoid biosynthesis enzyme and a selectable marker or screenable marker).

Vectors used for the transformation of lettuce cells are not limited so long as the vector can express an inserted DNA in the cells. For instance, vectors comprising promoters for constitutive gene expression in lettuce cells (e.g., cauliflower mosaic virus 35S promoter) and promoters inducible by exogenous stimuli can be used. Some examples of suitable vectors include a binary agrobacterium vector with a GUS reporter gene for plant transformation. The lettuce cell into which the vector is to be introduced includes various forms of lettuce cells, such as cultured cell suspensions, protoplasts, leaf sections, and callus. A vector can be introduced into lettuce cells by known methods, such as the polyethylene glycol method, polycation method, electroporation, Agrobacterium-mediated transfer, particle bombardment and direct DNA uptake by protoplasts.

In some embodiments, the plant transformation vector includes a selectable marker. In particular embodiments, the selectable marker is selected from a biocide resistance marker, an antibiotic resistance marker, or an herbicide resistance marker.

In some embodiments, the system of the present disclosure further comprises a screenable marker. In particular embodiments, the screenable marker is selected from a β-glucuronidase or uidA gene (GUS), an R-locus gene, a β-lactamase gene, a luciferase gene, a xylE gene, an amylase gene, a tyrosinase gene, and an α-galactosidase gene.

In some embodiments, the plant transformation vector is derived from a plasmid of Agrobacterium tumefaciens. In certain embodiments, the vector is derived from a Ti plasmid of Agrobacterium tumefaciens. In certain embodiments, the vector is derived from a Ri plasmid of Agrobacterium rhizogenes. Agrobacterium-mediated transfer is a widely applicable system for introducing gene loci into plant cells. Modern Agrobacterium transformation vectors are capable of replication in E. coli as well as Agrobacterium, allowing for convenient manipulations (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti genes can be used for transformation.

Protocols and methods for transformation via Agrobacterium-mediated plant integrating vectors to introduce DNA into lettuce plant cells have been established (Fraley et al., 1985; U.S. Pat. No. 5,563,055). For example, U.S. Pat. No. 5,349,124 describes a method of transforming lettuce plant cells using Agrobacterium-mediated transformation. By inserting a chimeric gene having a DNA coding sequence encoding for the full-length Bacillus thuringiensis (Bt) toxin protein that expresses a protein toxic toward Lepidopteran larvae, for example, caterpillar, this methodology resulted in lettuce having resistance against such insects.

Microprojectile bombardment techniques are widely applicable, and may be used to transform virtually any plant species. Examples involving microprojectile bombardment transformation with lettuce can be found in, for example, Elliott et al. 2004; Phys. Rev. Lett. 92, 095501.

### Transgenic Lettuce Cells and Transgenic Lettuce Plants

In some embodiments, disclosed herein is a transgenic lettuce that is transformed with one or more of the polynucleotides and/or expression cassettes described herein. As described herein, a transgenic lettuce cell can be a part of a lettuce plant. In some embodiments, disclosed herein is a transgenic lettuce cell transformed with the one or more polynucleotides and/or expression cassettes described herein. In some embodiments, the transgenic lettuce comprises the transgenic lettuce cell. In some embodiments, the transgenic lettuce or lettuce cell is a lettuce seed. In certain embodiments, the present disclosure provides a lettuce seed that comprises a system as described herein.

In some embodiments, the transgenic lettuce cell, transgenic lettuce, or transgenic lettuce seed of the present disclosure displays enhanced production of one or more polyphenols or derivatives thereof. In some embodiments, the enhanced production comprises increased production of the one or more polyphenols or derivatives thereof, relative to a control lettuce cell or control lettuce. In some embodiments, the enhanced production modification of the one or more polyphenols or derivatives thereof, relative to a control lettuce cell or control lettuce. In some embodiments, the one or more polyphenols or derivatives thereof are selected from chlorogenic acids, or derivatives thereof, such as 3-O-caffeoylquinic acid (3-CQA), 4-O-caffeoylquinic acid (4-CQA), 5-O-caffeoylquinic acid (5-CQA), 3,4-dicaffeoylquinic acid (3,4-diCQA), chicoric acid; quercetin and water-soluble quercetin derivatives, such as quercetin-3-O-glucoside (Q3G) and quercetin-3-O-malonylglucoside (Q3MG); other flavonoids such as apigenin and derivatives, luteolin and derivatives, chrysoeriol and derivatives, myricetin and derivatives; and anthocyanins such as cyaniding 3-malonyl-glucoside, cyandidin-3-O-glucoside and analogues. In some embodiments, the one or more polyphenols or derivatives thereof comprises quercetin-3-O-malonylglucoside (Q3MG). In some embodiments, the one or more polyphenols or derivatives thereof comprises 5-O-caffeoylquinic acid (5-CQA).

In certain embodiments, the polyphenols or derivatives thereof are selected from chlorogenic acids and quercetin. In some particular embodiments, the one or more polyphenols or derivatives thereof comprise 5-O-caffeoylquinic acid (5-CQA), 4-O-caffeoylquinic acid (4-CQA), 3-O-caffeoylquinic acid (3-CQA), 3,4-dicaffeoylquinic acid (3,4-diCQA), chicoric acid, quercetin, quercetin-3-O-malonylglucoside (Q3MG), and quercetin-3-O-glucoside (Q3G).

In some embodiments, the lettuce described herein is a lettuce cultivar with red leaves from a general lettuce type. In some embodiments, the lettuce of the present disclosure, wherein the general lettuce type is selected from loose leaf, oakleaf, romaine, butterhead, iceberg, and summer crisp lettuces. In some embodiments, the lettuce is a red leaf lettuce cultivar. In some embodiments, the red leaf lettuce cultivar is selected from Lollo Rossa, New Red Fire Lettuce, Red Sails Lettuce, Redina Lettuce, Galactic Lettuce, Batavian lettuce, and Benito Lettuce. In some embodiments, the lettuce is Annapolis, Lettuce, Hongjil Lettuce, Red Fire Lettuce, Jinluck Lettuce, Dazzler Lettuce, Seoul Red Lettuce, Revolution Lettuce, Cherokee Lettuce, Valerial Lettuce, OOC 1441 Lettuce, Impuls Lettuce, Red Mist Lettuce, Red Salad Bowl Lettuce, Red Tide Lettuce, Bellevue Lettuce, Outredgeous Lettuce, Pomegranate Crunch Lettuce, Vulcan Lettuce, Cantarix Lettuce, Breen Lettuce, Rouge D'Hiver Lettuce, Oscarde Lettuce, Blade Lettuce, Spock Lettuce, Edox Lettuce, Fortress Lettuce, Stanford Lettuce, Scaramanga Lettuce, or Rutgers Scarlet Lettuce.

In some embodiments, the transgenic lettuce cell comprises a suspension culture plant cell. In particular embodiments, the suspension culture plant cell is a cell of red leaf lettuce.

### Methods of Producing a Transgenic Plant Cell or Transgenic Plant

In some aspects, provided herein are methods of producing a transgenic lettuce that is capable of synthesizing one or more polyphenols. In some embodiments, the method includes: introducing into a lettuce cell a system, transgene, or expression cassette of the present disclosure to produce a transformed lettuce cell; culturing the transformed lettuce cell under conditions sufficient to allow development of a lettuce cell culture comprising a plurality of transformed lettuce cells; screening the transformed lettuce cells for expression of a polypeptide encoded by the system, transgene, or expression cassette; and selecting from the lettuce cell culture a transformed lettuce cell that expressed the polypeptide. In some embodiments, the transformation is performed with a protoplast, electroporation, agitation with silicon carbide fibers, Agrobacterium-mediated transformation, or by acceleration of DNA-coated particles. In some embodiments, the lettuce cell is transformed using Agrobacterium-mediated transformation and the plant transformation vector comprises an Agrobacterium vector. In some embodiments, selection of a transformed cell is based on detection of expression of a screenable marker. In some embodiments, the transformation can be stable transformation or transient transformation.

Various methods can be used to introduce a sequence of interest into a plant or plant part. “Introduce” or “introducing” is intended to mean presenting to the plant, plant cell or plant part the polynucleotide or polypeptide in such a manner that the sequence gains access to the interior of a cell of the plant. The methods of the present disclosure do not depend on a particular method for introducing a sequence into a plant or plant part, only that the polynucleotide or polypeptides gains access to the interior of at least one cell of the plant. Methods for introducing polynucleotide or polypeptides into plants are known in the art including, but not limited to, stable transformation methods, transient transformation methods, and virus-mediated methods.

“Stable transformation” is intended to mean that a polynucleotide integrates into the genome of the plant or integration of the polynucleotide into the genome of a plastid (i.e., the chloroplast, amyloplasts, chromoplasts, statoliths, leucoplasts, elaioplasts, and proteinoplasts), and the polynucleotide is capable of being inherited by the progeny of the plant. “Transient transformation” is intended to mean that a polynucleotide is introduced into the plant and does not integrate into the genome of the plant. Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, e.g., U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips

(Springer-Verlag, Berlin); McCabe et al. (1988) Biotechnology 6:923-926); and Lecl transformation (WO 00/28058). See also Weissinger et al. (1988) Ann. Rev. Genet. 22:421-477; Sanford et al. (1987) Particulate Science and Technology 5 27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311: 763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4: 1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In certain embodiments, the transforming is by Agrobacterium-mediated transformation and the plant transformation vector comprises an Agrobacterium vector. In particular embodiments, the Agrobacterium vector comprises a Ti plasmid or an Ri plasmid. Agrobacterium-mediated transfer is an established method in the art for introducing gene loci into plant cells. DNA can be introduced into whole plant tissues, thereby bypassing the need for regeneration of an intact plant from a protoplast. Agrobacterium transformation vectors are capable of replication in E. coli as well as Agrobacterium, allowing for convenient manipulations (Klee et al. 1985. Bio. Tech. 3(7):637-342). Moreover, vectors for Agrobacterium-mediated gene transfer have improved the arrangement of genes and restriction sites in the vectors to facilitate the construction of vectors capable of expressing various polypeptide coding genes. Such vectors have convenient multi-linker regions flanked by a promoter and a polyadenylation site for direct expression of inserted polypeptide coding genes. Additionally, Agrobacterium containing both armed and disarmed genes can be used for transformation.

In certain embodiments, the lettuce cell or lettuce plant is transformed using Agrobacterium tumefaciens Ti-plasmid-mediated transformation with the plant expression vector pSCP-ME (SignalChem). pSCP-ME is a binary vector for high-level expression of a foreign gene in dicotyledonous plants carrying the constitutive SCP promoter and a chimeric terminator. All the transgenes maybe cloned into pSCP-ME for transient or stable transformation.

### Methods of Producing Lettuce Polyphenols

In some aspects, provided herein are methods of producing one or more polyphenols or derivatives thereof. In some embodiments, the method producing one or more polyphenols or derivatives thereof comprising administering at least one eustressor/elicitor, or a homologue, isomer or derivative thereof disclosed herein to a lettuce plant or cell, thereby increasing the production of polyphenols in lettuce plant or cell. In certain embodiments, the at least one eustressor/elicitor is selected from: indole-3-acetic acid (IAA), naphthalene acetic acid (NAA), oxalic acid, benzothiadiazole (BTH), 2,4-dichlorophenoxy acetic acid (2,4-D), arachidonic acid (AA), salicylic acid (SA), methyl jasmonate (MJ), harpin protein (HP), or any combination or mixture thereof.

In some embodiments, the method of producing one or more polyphenols or derivatives thereof comprise culturing a transgenic lettuce cell or cultivating a transgenic lettuce, or lettuce seed of the present disclosure under conditions sufficient to produce the one or more polyphenols or derivatives thereof. In some embodiments, the transgenic lettuce cell, transgenic lettuce, or lettuce seed comprises an expression cassette comprising a heterologous expression control sequence operably linked to at least one polynucleotide encoding one or more proteins that increase the production of polyphenols in lettuce. In certain embodiments, the expression cassette comprises a polynucleotide encoding a malonate-CoA ligase. In some embodiments, the malonate-CoA ligase is AAE13. In some embodiments, the expression cassette comprises a polynucleotide encoding a MYB transcription factor. In some embodiments, the MYB transcription factor is a AtMYB12 transcription factor. In some embodiments, the expression cassette comprises a polynucleotide encoding an enzyme of the phenylpropanoid pathway. In particular embodiments, the enzymes of the phenylpropanoid pathway are selected from: phenylalanine ammonia-lyase (PAL), cinnamic acid 4-hydroxylase (C4H), and 4-coumaric acid: CoA ligase (4CL), or any combination thereof. In certain embodiments, the expression cassette comprises a polynucleotide encoding an enzyme of the chlorogenic acid pathway. In particular embodiments, the enzymes of the chlorogenic acid pathway are selected from: hydroxycinnamoyl CoA: quinate hydroxycinnamoyl transferase (HQT), p-coumaroyl-3-hydroxylase (C3H), and caffeoyl-CoA-3-O-methyltransferase (CCoAMT), or any combination thereof. In certain embodiments, the expression cassette comprises a polynucleotide encoding an enzyme of the flavonoid pathway. In particular embodiments, the enzymes of the flavonoid pathway are selected from: chalcone synthase (CHS), chalcone isomerase (CHI), flavanone 3-hydroxylase (F3H), and flavonol synthase (FLS), flavonoid 3′-hydroxylase (F3′H), p-coumarate 3-hydroxylase (C3H), cinnamate 4-hydroxilase (C4H), 4-hydroxycinnamoyl-CoA ligase (4CL), hydroxycinnamoyl-CoA shikimate/quinate hydroxycinnamoyl transferase (HCT), hydroxycinnamoyl-CoA quinate hydroxycinnamoyl transferase (HQT), or any combination thereof. In certain embodiments, the expression cassette comprises a polynucleotide encoding a cytochrome P450 3A4, CYP oxidoreductase, and UDP-glucuronosyltransferase, or any combination thereof

In some embodiments, the one or more polyphenols or derivatives thereof is selected from: chlorogenic acid or derivatives thereof, chicoric acid, and/or water-soluble quercetin derivative. In some embodiments, the chlorogenic acid is 3-O-caffeoylquinic acid (3-CQA), 4-O-caffeoylquinic acid (4-CQA), and/or 5-O-caffeoylquinic acid (5-CQA); the chicoric acid is (2R,3R)-O-dicaffeoyltartaric acid; and/or wherein the water-soluble quercetin derivative is quercetin-3-O-glucoside (Q3G) and/or quercetin-3-O-malonylglucoside (Q3MG). In some embodiments, the increased production of polyphenols is quantified by LC-MS. In some embodiments, the increased production of polyphenols is quantified by HPLC.

### Extracts and Food Products

In certain embodiments, the present disclosure provides an extract of the lettuce cell, transgenic lettuce, or lettuce seed of the present disclosure that comprise an increased amount of one or more polyphenols or derivatives thereof compared to controls. In some embodiments, the extract of the present disclosure is red lettuce extract SLC1021. In some embodiments, the extract comprises water and ethanol and lettuce components that are soluble therein. In some embodiments, the extract comprises about 2% chlorogenic acids, 2% chicoric acid, and 2% anthocyanins and about 3.5% quercetin (w/w).

In some embodiments, the present disclosure provides a method of making a lettuce extract comprising mixing a lettuce sample with a solvent and separating the liquid phase from the solid phase. In some embodiments, the solvent is a food grade solvent. In certain embodiments, the solvent is ethanol. The lettuce sample may be fresh, frozen, or dehydrated. In some embodiments, the ratio of lettuce to solvent (g/mL) is 1:10, 1:5, 2:5, 3:5, 4:5, or 1:1. In certain embodiments, the ratio of lettuce to solvent (g/mL) is 2:5. In some embodiment, the method of making a lettuce extract comprising freezing a lettuce sample, grinding the frozen lettuce sample, mixing the lettuce sample with ethanol at a 2:5 ratio (g/mL), and separating the liquid phase from the solid phase.

In some embodiments, the lettuce extract prevents or reduces symptoms of viral or bacterial infection, diabetes, cardiovascular diseases, neurodegenerative diseases, including memory and eyesight loss, inflammation, and cancer. In some embodiments, the lettuce extract is an antioxidant that provides an anti-inflammatory, anticancer, antimicrobial, antiallergic, antiviral, antithrombotic, and/or hepatoprotective effect. In some embodiments, the lettuce extract inhibits or reduces viral replication, reduces inflammation, improves visual acuity, modulates the immune response, reduces obesity and diabetes, reduces blood glucose levels, or any combination thereof.

In some embodiments, disclosed herein is a food product containing lettuce or lettuce parts described in the instant disclosure. A “food product” as used herein includes a lettuce plant part described herein and/or an extract from a lettuce plant part described herein. The food product may be fresh or processed, e.g., canned, steamed, boiled, fried, blanched and/or frozen. Moreover, the food products of the present disclosure are not particularly limited. For instance, the present disclosure is applicable to the preparation of food products for consuming lettuces such as: salad, sandwich, in soup, as juice, as lettuce wraps, seared or sautéed, grilled, braised, layered into spring rolls and wraps, with rice and/or noodle bowls, and as sauce. In some embodiments, the food product is for mammals. In some embodiments, the food product is for a human.

In some embodiments, the food product prevents or reduces symptoms of viral or bacterial infection, diabetes, cardiovascular diseases, neurodegenerative diseases, including memory and eyesight loss, inflammation, and cancer. In some embodiments, the food product is an antioxidant that provides an anti-inflammatory, anticancer, antimicrobial, antiallergic, antiviral, antithrombotic, and/or hepatoprotective effect. In some embodiments, the food product inhibits or reduces viral replication, reduces inflammation, improves visual acuity, modulates the immune response, reduces obesity and diabetes, reduces blood glucose levels, or any combination thereof.

### Methods of Treating Viral Infection

In some embodiments, disclosed herein is a method for treating a viral infection comprising administering an effective amount of the extract or the food product of the present disclosure to a patient in need thereof. In some embodiments, the virus is a coronavirus (e.g., COVID-19, SARS, MERS), influenza A (Flu A), respiratory syncytial virus (RSV), Zika virus, Dengue virus (DENV2). In certain embodiments, phenolic compounds present in the extract or food product inhibit and/or interfere with the activity of viral proteins. As used herein, the term “inhibit” refers to reduction or prevention of at least one activity of a target protein. The activity can be inhibited and/or reduced by at least 10%, 20%, 30%, 40%, 50%, 60%, 70%, 80%, 90%, 95%, 98%, 99%, or 100%, as measured by the methods disclosed herein or known in the art. In some embodiments, the method for treating a viral infection comprises an extract that is red lettuce extract SLC1021. In some embodiments, the concentration of the extract is about 10 μg/mL-200 μg/mL, 10 μg/mL-150 μg/mL, 10 μg/mL-100 μg/mL, 10 μg/mL-90 μg/mL, 10 μg/mL-80 μg/mL, 10 μg/mL-70 μg/mL, 10 μg/mL-60 μg/mL. In some embodiments, the concentration of SLC1021 is greater than about 1 μg/mL, 2 μg/mL, 3μg/mL, 4μg/mL, 5 μg/mL, 6 μg/mL, 7 μg/mL, 8 μg/mL, 9 μg/mL, 10 μg/mL, 20 μg/mL, 30 μg/mL, 40 μg/mL, 50 μg/mL, 60 μg/mL, 70 μg/mL, 80 μg/mL, 90 μg/mL, 100 μg/mL, 120 μg/mL, 140 μg/mL, 160 μg/mL, 180 μg/mL, 200 μg/mL, 250 μg/mL, 300 μg/mL350 μg/mL, 400 μg/mL, 450 μg/mL, or 500 μg/mL. In any of the embodiments disclosed here, the patient can be a human.

In some embodiments, is a method for treating a viral infection by coronavirus (e.g., COVID-19, SARS, MERS). In some embodiments, the coronavirus is a severe acute respiratory syndrome coronavirus 2 (SARS-CoV-2). In some embodiments, the SARS-CoV-2 causes coronavirus disease 2019 (COVID-19). In some embodiments, the method for treating coronavirus infection comprises administering an effective amount of the extract or the food product of the present disclosure to a patient infected with a coronavirus, wherein the activity of 3-chymotrypsin-like protease (3CL^{pro}) is inhibited. The 3-chymotrypsin-like protease (3CL^{pro}) is a cysteine protease that plays an important role in proteolytic processing of viral polyproteins, thought to be necessary proteins for viral replication and function.

In some embodiments, the method for treating a coronavirus infection comprises administering an effective amount of the extract or the food product of the present disclosure to a patient infected with a coronavirus, wherein the activity of RNA-dependent RNA polymerase (RdRp) is inhibited and/or reduced. The RNA-dependent RNA polymerase (RdRp), also known as nsp12, mediates viral replication by catalyzing the replication of RNA from an RNA template. RdRp is the core component of a replication/transcription catalytic complex of viral nonstructural proteins (nsp). Due to its vital role for the life cycle of RNA viruses, RdRp has been proposed to be the target of a class of antiviral drugs that are nucleotide analogs, including remdesivir.

In some embodiments, the method for treating a coronavirus infection comprises administering an effective amount of the extract or the food product of the present disclosure to a patient infected with a coronavirus, wherein the activity of RNA helicase and triphosphatase (nsp13) is inhibited. The RNA helicase (nsp13) of SARS-CoV-2 is a superfamily 1 helicase that shares 99.8% sequence identity and a strikingly conserved overall architecture with the SARS-CoV-1 nsp13. Like other coronaviruses, SARS-CoV-2 nsp13 exhibits multiple enzymatic activities. Nsp13 is thought to be a necessary enzyme in viral replication, and frequently interacts with the host immune system.

In some embodiments, the method for treating a coronavirus infection comprises administering an effective amount of the extract or the food product of the present disclosure to a patient infected with a coronavirus, wherein the binding of the Spike protein to ACE2 is inhibited. In certain embodiments, the Spike protein is 2019-nCoV Spike protein. In some embodiments, the interaction of the Spike protein receptor binding domain (RBD) with ACE2 is inhibited.

In some embodiments, is a method for treating a viral infection by influenza A (Flu A) comprising administering an effective amount of the extract or the food product of the present disclosure to a patient in need thereof. In some embodiments, the method for treating the Flu A infection comprises an extract that is red lettuce extract SLC1021. In some embodiments, the concentration of the extract is about 1-100 μg/mL. In some embodiments, the concentration of the extract is about 10.3 μg/mL, 30.9 μg/mL, or 92.6 μg/mL.

In some embodiments, is a method for treating a viral infection by respiratory syncytial virus (RSV) comprising administering an effective amount of the extract or the food product of the present disclosure to a patient in need thereof. In some embodiments, the concentration of the extract is about 1-400 μg/mL. In some embodiments, the concentration of the extract is about 4.1 μg/mL, 12.43 μg/mL, 37 μg/mL, 111 μg/mL, or 333 μg/mL.

In some embodiments, is a method for treating a viral infection by Zika virus comprising administering an effective amount of the extract or the food product of the present disclosure to a patient in need thereof. In some embodiments, the concentration of the extract is about 1-1000 μg/mL.

In some embodiments, is a method for treating a viral infection by Dengue virus (DENV2) comprising administering an effective amount of the extract or the food product of the present disclosure to a patient in need thereof. In some embodiments, the concentration of the extract is about 1-1000 μg/mL.

### Method of Treating Cancer

In some embodiments, disclosed herein is a method for treating a cancer comprising administering an effective amount of the extract or the food product of the present disclosure to a patient in need thereof. In some embodiments, the cancer is a leukemia, lymphoma, breast cancer, or prostate cancer. In certain embodiments, phenolic compounds present in the extract or food product have a cytotoxic effect on cancer cells. In some embodiments, treatment results in at least one of: tumor regression, a decreased rate of tumor progression, a reduced level of a cancer biomarker, reduced symptoms associated with cancer, a prevention or delay in metastasis, or clinical remission. In some embodiments, the method for treating a cancer comprises an extract that is red lettuce extract SLC1021. In some embodiments, the concentration of the extract is about 0.1 mg/mL-5 mg/mL, 0.2 mg/mL-4 mg/mL, 0.2 mg/mL-3 mg/mL, 0.3 mg/mL-3 mg/mL, 0.4 mg/mL-3 mg/mL, 0.5 mg/mL-3 mg/mL, 0.4 mg/mL-2.5 mg/mL, 0.4 mg/mL-2.0 mg/mL, or 0.4 mg/mL-1.6 mg/mL. In some embodiments, the concentration of the extract is greater than about 0.1 mg/mL, 0.2 mg/mL, 0.3 mg/mL, 0.4 mg/mL, 0.5 mg/mL 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, or 2.0 mg/mL. In certain embodiments, the concentration of the extract is about 0.02 mg/mL, 0.06 mg/mL, 0.19 mg/mL, 0.56 mg/mL, 1.67 mg/mL, or 5 mg/mL.

### Methods for Treating Inflammatory Conditions or Diseases

In some embodiments, disclosed herein is a method for treating an inflammatory condition or disease, comprising administering an effective amount of the extract or the food product of the present disclosure to a patient in need thereof. In certain embodiments, phenolic compounds present in the extract or food product inhibit the production of inflammatory cytokines by immune cells. Examples of immune cells include monocytes, macrophages, dendritic cells, T cells, B cells, and natural killer cells. Examples, of inflammatory cytokines include IL-6 and TNFαIn some embodiments, the method for treating an inflammatory condition or disease comprises an extract that is red lettuce extract SLC1021. In some embodiments, the concentration of the extract is about 0.1 mg/mL-5 mg/mL, 0.2 mg/mL-4 mg/mL, 0.2 mg/mL-3 mg/mL, 0.3 mg/mL-3 mg/mL, 0.4 mg/mL-3 mg/mL, 0.5 mg/mL-3 mg/mL, 0.4 mg/mL-2.5 mg/mL, 0.4 mg/mL-2.0 mg/mL, or 0.4 mg/mL-1.6 mg/mL. In some embodiments, the concentration of the extract is greater than about 0.1 mg/mL, 0.2 mg/mL, 0.3 mg/mL, 0.4 mg/mL, 0.5 mg/mL 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, or 2.0 mg/mL. In certain embodiments, the concentration of the extract is about 0.02 mg/mL, 0.06 mg/mL, 0.19 mg/mL, 0.56 mg/mL, 1.67 mg/mL, or 5 mg/mL.

### Methods for Inhibiting the Production of Reactive Oxygen Species (ROS)

In some embodiments, disclosed herein is a method for inhibiting the production of reactive oxygen species (ROS), comprising administering an effective amount of the extract or the food product of the present disclosure to a patient in need thereof. In certain embodiments, phenolic compounds present in the extract or food product inhibit the production of ROS in a cell. Examples of ROS include nitric oxide. In some embodiments, the method for inhibiting the production of ROS comprises an extract that is red lettuce extract SLC1021. In some embodiments, the concentration of the extract is about 0.1 mg/mL-5 mg/mL, 0.2 mg/mL-4 mg/mL, 0.2 mg/mL-3 mg/mL, 0.3 mg/mL-3 mg/mL, 0.4 mg/mL-3 mg/mL, 0.5 mg/mL-3 mg/mL, 0.4 mg/mL-2.5 mg/mL, 0.4 mg/mL-2.0 mg/mL, or 0.4 mg/mL-1.6 mg/mL. In some embodiments, the concentration of the extract is greater than about 0.1 mg/mL, 0.2 mg/mL, 0.3 mg/mL, 0.4 mg/mL, 0.5 mg/mL 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, or 2.0 mg/mL. In certain embodiments, the concentration of the extract is about 0.02 mg/mL, 0.06 mg/mL, 0.19 mg/mL, 0.56 mg/mL, 1.67 mg/mL, or 5 mg/mL.

### EXAMPLES

The following examples are offered by way of illustration and not by way of limitation.

### Example 1

### Enhancement of Polyphenol Production in Red Lettuce Using Eustressor/Elicitors

This example demonstrates increased in production of polyphenols in red leaf lettuce when treating with biotic/abiotic eustressors/elicitors.

### Plant Materials, Growth Conditions and Eustressor/Elicitor Treatments

Lettuce plants (Lactuca sativa) of red varieties were grown in a lab greenhouse with an average photoperiod of 12 h/day, at 25-28° C., 40-60% relative humidity. Abiotic eustressors/elicitors used were indole-3-acetic acid (IAA), naphthalene acetic acid (NAA), oxalic acid, benzothiadiazole (BTH); 2,4-dichlorophenoxy acetic acid (2,4-D), arachidonic acid (AA), salicylic acid (SA), and methyl jasmonate (MJ) at 5, 10, 15, 45, and 90 μM. Biotic eustressors/elicitors used were harpin protein (HP), chitosan, Burdock fructooligosaccharide (BFO), Reynoutria sachalinensis extract, and sea weed extract at 30, 60,120, and 1000 mg/L. All eustressors were dissolved in deionized water (non-water soluble eustressors were previously dissolved in 1 mL of ethanol). A group of samples and water with only 1 mL of ethanol were added. Control samples with no treatment were added. Eustressor/elicitor treatments were applied on the 14th preharvest day on red lettuces. Each experimental unit consisted of five lettuces randomly selected and assigned to one treatment. Each sample was treated by rooting absorption or foliar aspersion, with 3 sprays of each elicitor (approximately 1.70 mL). Lettuce samples were harvested at 50 d.

### Extraction and Quantification

Major health beneficial polyphenols were characterized and quantified in treated and untreated (control) red lettuces after extracting samples with 50% ethanol. Generally, two grams of the sample were frozen with liquid nitrogen, ground, and mixed with 5 mL of ethanol. The sample/ethanol mixture were shaken 4 hours at room temperature and centrifuged at 5000×g for 10 min (4° C.). The supernatant was collected, filtered, and subjected to LC-MS analysis.

### Results

The enhanced production of polyphenols was confirmed using LC/MS/UV.

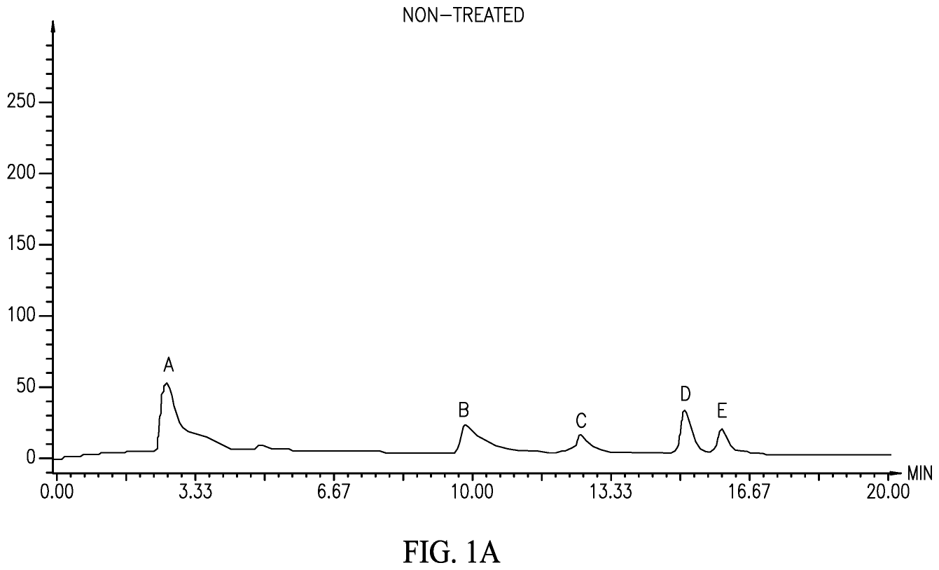

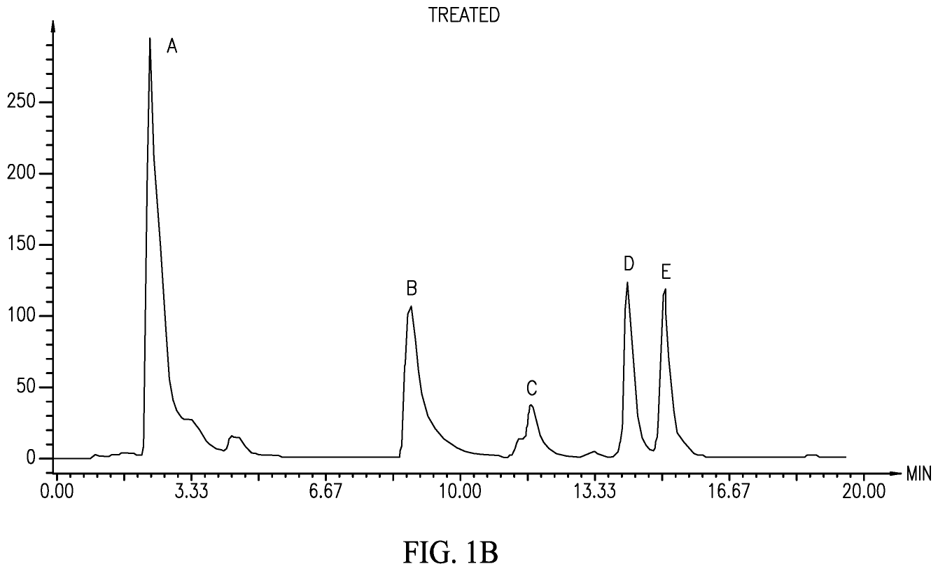

As shown in FIG. 1, the chromatograms of bioactive components enhancement by genomics-based technologies confirm production of specific metabolites in red lettuce treated with biotic or abiotic eustressors. Polyphenols chlorogenic acid (3-CQA); chicoric acid; 3,4-dicaffeoylquinic acid (3,4-diCQA); Quercetin-3-O-glucoside (Q3G), Quercetin-3-O-malonylglucoside (Q3MG), show enhanced production in treated lettuce compared to non-treated lettuce control.

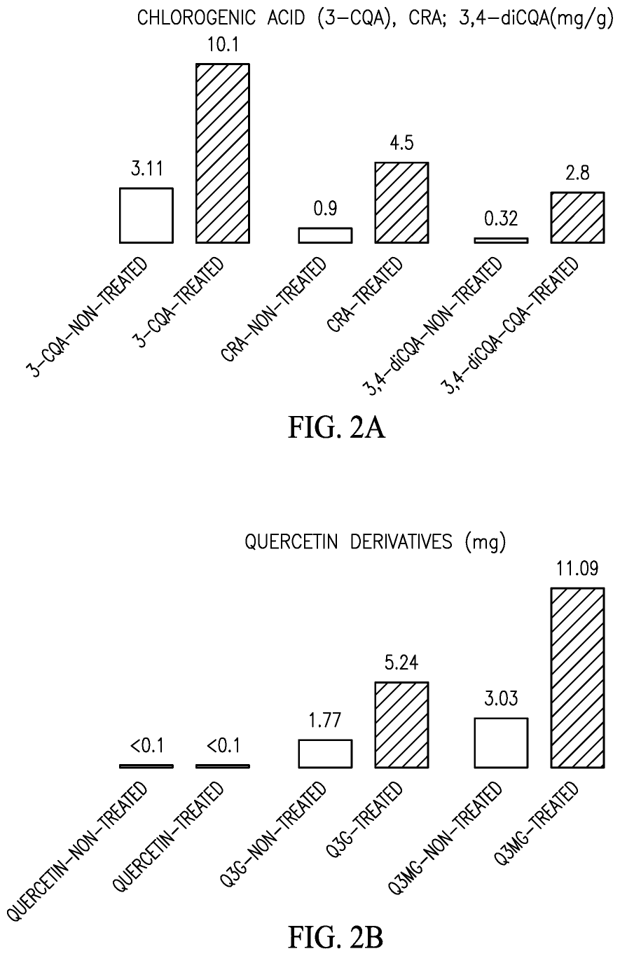

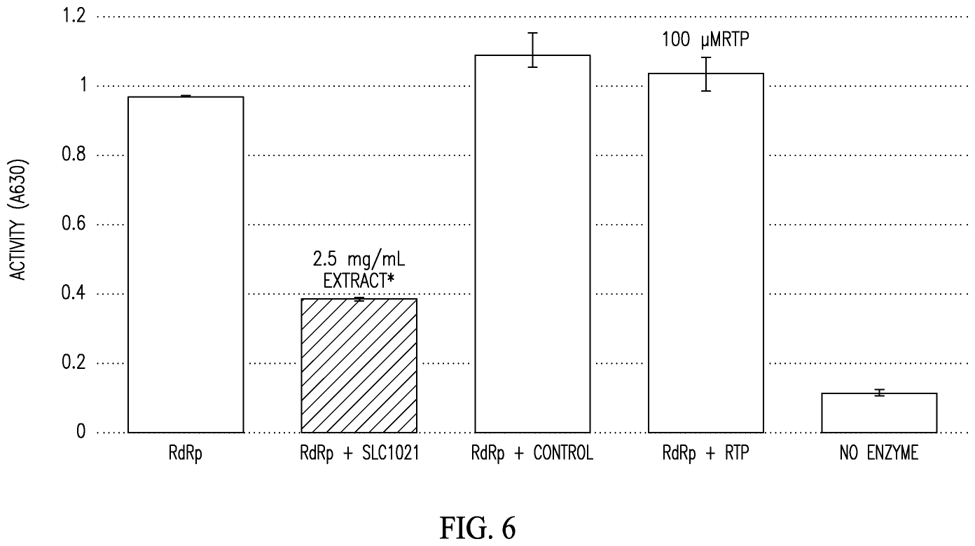

As shown in FIGS. 2A-2B, the production of chlorogenic acids (FIG. 2A) and the water-soluble quercetin derivatives (FIG. 6B) were increased by 3- to 9-fold in red lettuce treated with eustressors/elicitors. Chlorogenic acid and derivatives (3-CQA, chicoric acid, and 3,4-diCQA) and quercetin derivatives (Q3G and Q3MG) show enhanced production in treated lettuce compared to non-treated lettuce control.

These results demonstrate that treatment with abiotic and/or biotic eustressors increase in vivo polyphenol production in red lettuce. The combination of elicitor/eustressors treatments could show an additive or synergetic response.

### Example 2

### Enhancement of Polyphenol Production in Red Lettuce by Regulation of Genes of the Main Phenylpropanoid Pathway

This example shows enhancement of polyphenols by regulation of genes of a primary phenylpropanoid pathway. More specifically, this example increases the polyphenol content in red lettuce by overexpression of AAE13 and ATMYB12 as a representative example of in vivo production of bioactive molecules in an edible vegetable by up-regulation of the primary phenylpropanoid biosynthetic pathway using the present disclosure's proprietary genomics-based technologies (e.g., system) to enhance production of downstream metabolites.

A high-efficiency platform for transient expression and stable transformation of plant suspension cells technologies developed by SignalChem was used. Specifically, Agrobacterium tumefaciens Ti-plasmid-mediated was transformed with the plant expression vector pSCP-ME (SignalChem), a binary vector for high-level expression of a foreign gene in dicotyledonous plants carrying the constitutive SCP promoter and a chimeric terminator. To engineer the biosynthesis of malonyl-CoA and increase building blocks for the health beneficial polyphenol synthesis, the transgenes AAE13 (malonate-CoA ligase) and AtMYB12 transcription factor were cloned into pSCP-ME for transient and stable transformation.

Overnight cultures of Agrobacterium strain AGL1 harboring the transgenes were transferred to a 1000 mL flask with 250 mL YEP medium supplemented with 100 mg/L of kanamycin, 50 mg/L of carbenicillin and 50 mg/L of rifampicin and grown for 4-8 hours until the optical density at 600 nm (0D600) reached approximately between 0.5 and 1. The cells were pelleted in a centrifuge at room temperature and resuspended in 45 mL of infiltration medium containing 5 g/L D-glucose, 10 mM IVIES, 10 mM MgC12 and 200 μM acetosyringone. Agroinfiltration method by vacuum infiltration was used for transient expression and stable transformation in red lettuce leaves.

### Results

The enhanced production of polyphenols was confirmed using LC/MS.

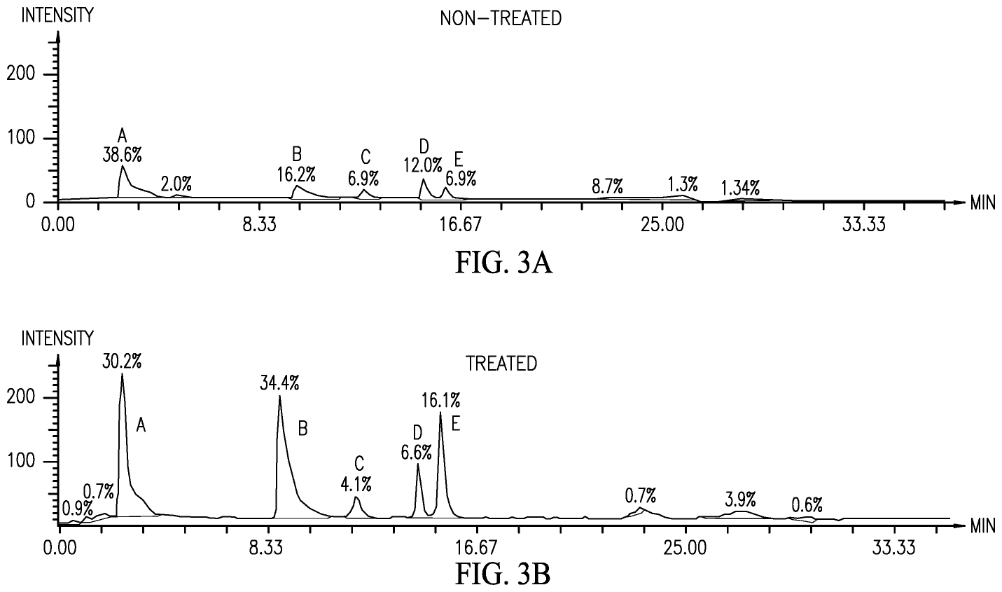

The accumulation of polyphenols was confirmed using LC/MS in 5-7 days after agroinfiltration. FIG. 3 shows a chromatograph demonstrating production of polyphenols by red lettuce leaf cells. The present disclosure demonstrates that infiltration of lettuce leaves with Agrobacterium carrying above genes was accomplished as described herein. The accumulation of polyphenols was confirmed using LC/MS in 5-7 days after agroinfiltration.

As shown in FIG. 3, chromatograms (HPLC-UV) of bioactive components enhancement by genomics-based technologies confirm production of specific metabolites in red lettuce treated with regulation of genes of the main phenylpropanoid pathway. Polyphenols 3-CQA, Chicoric acid, 3,4-Dicaffeoylquinic acid (3,4-diCQA), Quercetin-3-O-glucoside (Q3G), Quercetin-3-O-malonylglucoside (Q3MG) show enhanced production in treated lettuce compared to non-treated lettuce control.

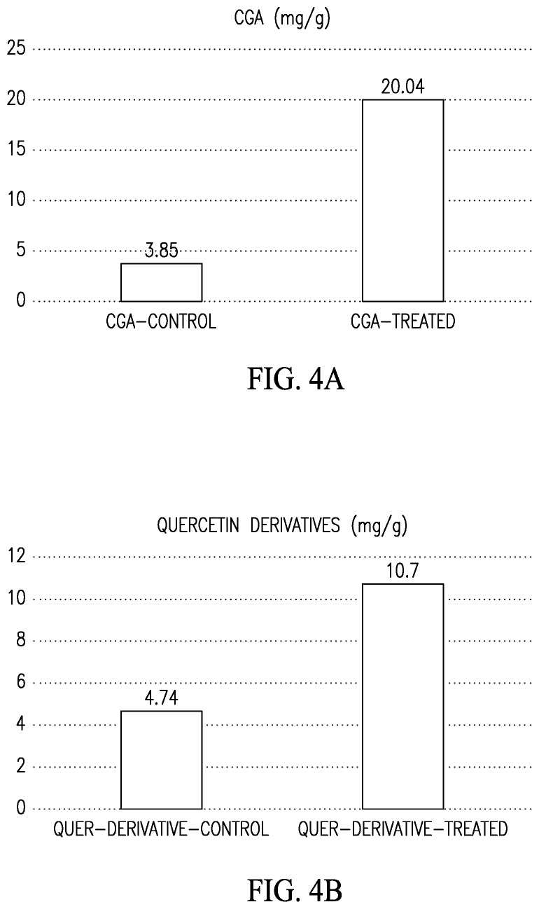

As shown in FIGS. 4A-4B, the production of chlorogenic acids (FIG. 4A) and the water-soluble quercetin derivatives (FIG. 4B) were significantly increased in red lettuce after the treatment by regulation of genes of the main phenylpropanoid pathway. Chlorogenic acids and derivatives thereof (3-CQA, chicoric acid, and 3,4-diCQA) and quercetin derivatives (Q3G and Q3MG) show enhanced production in treated lettuce compared to non-treated lettuce control.

These results demonstrate that regulation of genes of a primary phenylpropanoid pathway, such as by overexpression of AAE13 and ATMYB12, increase in vivo polyphenol production in red lettuce.

### Example 3

### Red Lettuce Extracts of the Present Disclosure Show Inhibition of Covid-19

The following examples demonstrate that the red lettuce extracts with high polyphenol contents from the present disclosure contain various biological activities.

To test for inhibition of SARS-CoV-2, COVID-19 virus proteins including 3-chymotrypsin-like protease (3CL^{pro}), RNA-dependent RNA polymerase (RdRp), and SARS-CoV-2 RNA helicase (nsp13) were expressed and purified. Enzyme inhibition assays were performed to confirm the activities of each purified protein. All enzymatic assays were based on spectrophotometric methods.

Treated red lettuce extract (SLC1021) was prepared using the methods as described in Examples 1 and 2. Major polyphenols were characterized and quantified with the LC-MS analysis. The extract (SLC1021) was tested in enzyme inhibition assays.

### Results

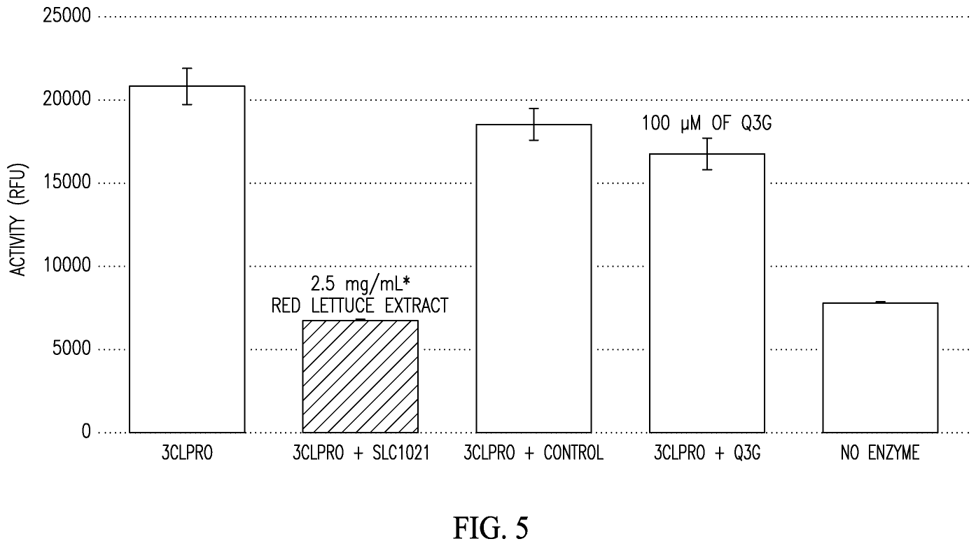

As shown in FIG. 5, treated red lettuce extract (SLC1021) shows inhibition of SARS-CoV-2 3-chymotrypsin-like protease (3CL^{pro}). Much stronger inhibitory effect of SLC1021 (Red lettuce extract) (3 CL^{pro}+SLC1021) was demonstrated when compared to the untreated plant extract (3CL^{pro}+control) or pure quercetin-3-O-glucoside (3CL^{pro}+Q3G).*: equivalent to 100 mM of quercetin derivatives in the plant extract.

As shown in FIG. 6, treated red lettuce extract (SLC1021) shows inhibition of SARS-CoV-2 RNA-dependent RNA polymerase (RdRp). Stronger inhibitory effect of SLC1021 (RdRp+SLC1021) was observed when compared to the untreated plant extract (RdRp+control) & metabolized remdesivir (RdRp+RTP).*: equivalent to 100 mM of quercetin derivatives in the plant extract.

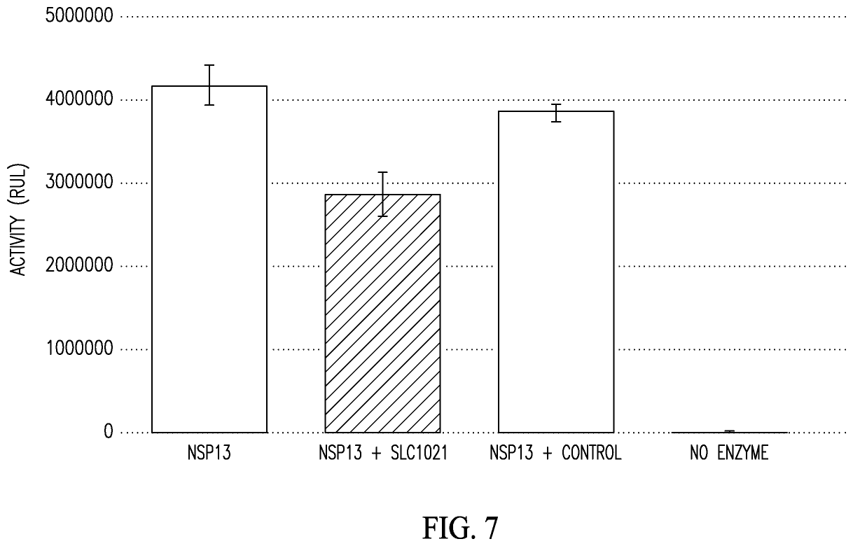

As shown in FIG. 7, treated red lettuce extract (SLC1021) shows inhibition of SARS-CoV-2 RNA helicase and triphosphatase (nsp13). Stronger inhibitory effect of SLC1021 (nsp13+SLC1021) was observed when compared to untreated plant extract (nsp13+control).*: equivalent to 100 mM of quercetin derivatives in the plant extract.

### Example 4

### Red Lettuce Extracts of the Present Disclosure Show Inhibition of Sars-Cov-2 Virus in Vero E6 Cells

Experiments were performed to test treated red lettuce extract (SLC1021) inhibition of SARS-CoV-2 virus-induced cytopathic effects (CPE) in Vero E6 cells. Experiments were also performed to assess treated red lettuce extract (SLC1021) effect on cell viability following SARS-CoV2 virus (SARS-CoV2_{USA/WA1/2020}) replication in Vero E6 cells. Treated red lettuce extract (SLC1021) was prepared using the methods as described in Examples 1 and 2. Major polyphenols were characterized and quantified with the LC-MS analysis.

Method: Virus-induced cytopathic effects (CPE) and cell viability following SARS CoV2 virus (SARS-CoV2_{USA/WA1/2020}) replication in Vero E6 cells were measured by neutral red dye. Cells were seeded in 96-well flat-bottom tissue culture plates and allowed to adhere overnight at 37° C. and 5% CO2 to achieve 80-100% confluence. Following incubation, diluted test compounds and virus diluted to a pre-determined titer to yield more than 80% cytopathic effect at 3 days post-infection were added to the plate. Following incubation at 37° C., 5% CO₂ for 3 days, plates were stained with neutral red dye for approximately 2 hours. Supernatant dye was removed, and wells rinsed with PBS, and incorporated dye was extracted in 50:50 Sorensen citrate buffer/ethanol for >30 minutes and the optical density was read on a spectrophotometrically at 540 nm. Percent CPE reduction of the virus-infected wells and the percent cell viability of uninfected drug control wells were calculated to determine the EC50 and TC50 values using four parameter curve fit analysis. The EC50 represent the concentration of test compound to inhibit CPE by 50%; The TC50 was the concentration that caused 50% cell death in the absence of virus.

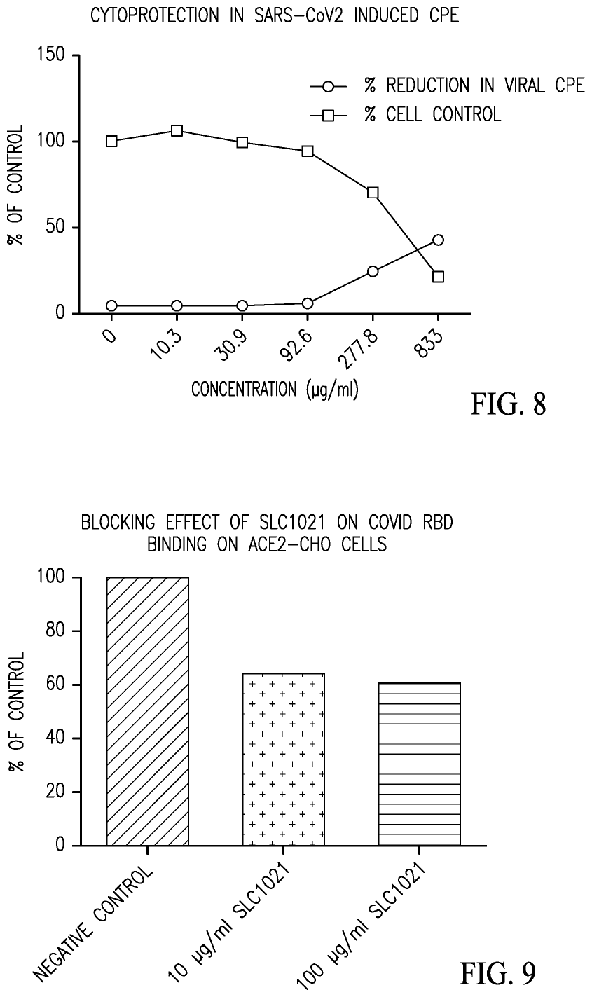

Results: SLC1021 showed a potential for cytoprotection from SARS-CoV2 induced cytopathic effect (CPE) in Vero E6 cells. A cytoprotection trend was demonstrated when the concentration of SLC1021 reached >92.6 μg/ml, although the EC50 did not reach 50% (FIG. 8).

### Example 5

### Blocking of Sars-Cov Spike Protein RBD Binding of ACE2-Cho Cells with SLC1021

Coronaviruses use the homotrimetic spike glycoprotein on the viral envelope to bind to their cellular receptors, e.g., ACE2. The spike glycoprotein comprises an S1 subunit and S2 subunit in each spike monomer, Coronaviruses binding to cellular receptors triggers a cascade of events that leads to the fusion between cell and viral membranes for cell entry. Therefore, binding to the ACE2 receptor is thought to be a critical initial step for SARS-CoV to enter into target cells. The receptor binding domain (RBD) is an important functional component within the S1 subunit that is responsible for binding of SARS-CoV-2 by ACE2 (Lan, J., Ge, J., Yu, J. et al. Nature 2020, 581, 215-220).

To demonstrate SLC1021 blocking of 2019-nCoV Spike protein RBD interaction with ACE2, human ACE2 stable cell line-CHO (SignalChem, A51C2-71C) was used for this assay. Treated red lettuce extract (SLC1021) was prepared using the methods as described in Examples 1 and 2. Major polyphenols were characterized and quantified with the LC-MS analysis.

Method: To demonstrate SLC1021 blocking of 2019-nCoV Spike protein RBD interaction with ACE2, human ACE2 stable cell line-CHO (SignalChem, A51C2-71C) was used for this assay. 2019-nCoV Spike protein RBD, His tag (SignalChem, C19SD-G241H), anti-2019-nCoV spike protein hIgG antibody (SignalChem, C19S1-61H) and mouse anti-human IgG BB700 (BD, 742235) were used according to manufacturer's instructions. Confirmation of successful binding of RBD to ACE2 was determined by staining with anti-spike protein hIgG and anti-human IgG via flow cytometry analysis. ACE2-CHO cells (target cells) were cultured according to manufacturer protocol. 10 μg/mL Spike protein RBD was pre-incubated with 100 μg/mL or 10 μg/mL of SLC1021 for 30 minutes and subsequently added to target cells. The target cells were incubated on ice for 1 h and then washed twice with PBS. Control cells were incubated with 10 μg/mL Spike protein RBD without SLC1021. Anti-spike protein hIgG at 5 μg/mL was added and incubated on ice for 1 h. The cells were washed twice with PBS and mouse anti-human IgG BB700 was added. The cells were incubated again on ice for 1 h. The cells were then washed twice with PBS and analyzed by flow cytometry using CytoFLEX (Beckman). Flow cytometry data were analyzed using FlowJo (BD Biosciences).

Results: The results demonstrate that SLC1021 reduced binding of the Spike protein RBD to ACE2-CHO cells compared to control (FIG. 9).

### Example 6

### The Cytoprotection of SLC1021 on Human Flu A and RSV Induced Cytopathic Effect (CPE) in RPMI2650 Cells

The cytoprotective effect of SLC1021 on RPMI2650 cells infected with human influenza virus (Flu A), Zika virus, Dengue virus (DENV2), or respiratory syncytia virus (RSV) in was evaluated. Treated red lettuce extract (SLC1021) was prepared using the methods as described in Examples 1 and 2. Major polyphenols were characterized and quantified with the LC-MS analysis.

Method: Inhibition of virus-induced cytopathic effects (CPE) and cell viability following human influenza virus (Flu_{APR834}) and respiratory syncytia virus type A (RSV_{A2}) replication in RPMI2650 cells, Zika and DENV2 virus in replication in Hub 7 cells, was measured by a chemiluminescenct endpoint (CellTiterGlo). Cells (5×10\hat{ }5 cells per well) were seeded in 96-well flat-bottom tissue culture plates and allowed to adhere overnight at 37° C. and 5% CO2. Following incubation, diluted test compounds and virus diluted to a pre-determined titer to yield at least 50% cell killing at 4 days

(FluA) or 80% cell killing at 5 days (RSV) post-infection were added to the plate. Following incubation at 37° C., 5% CO₂ for 4-5 days, cell viability was measured using a CellTiterGlo. Percent reduction of the virus-infected wells and the percent cell viability of uninfected drug control wells were calculated to determine the EC50 and TC50 values using four parameter curve fit analysis. The EC50 was the concentration of test compound to inhibit CPE by 50%; The TC50 was the concentration that caused 50% cell death in the absence of virus.

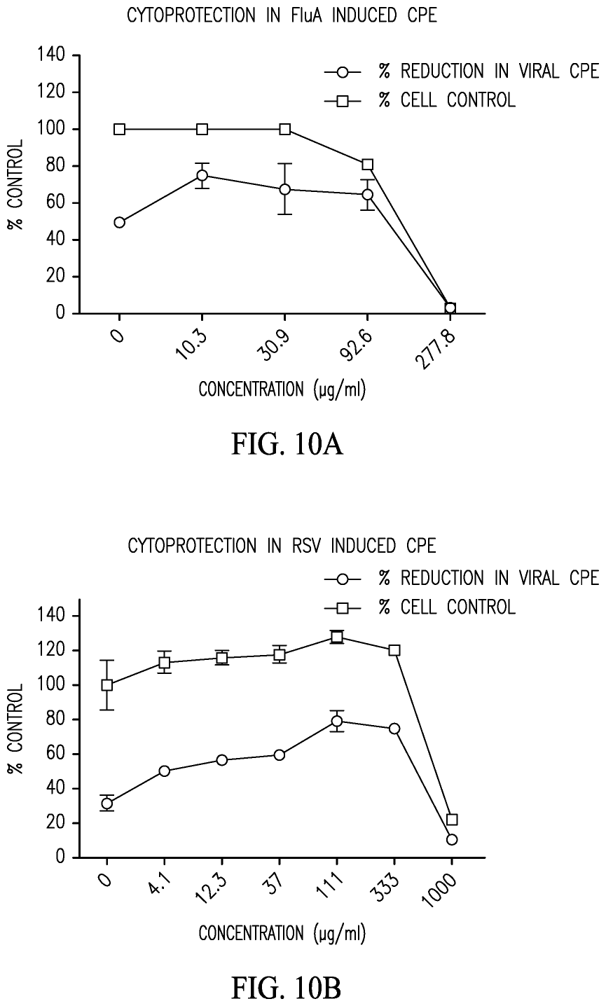

Results: SLC1021 demonstrated an inhibition of cytopathic effect in RPMI2650 cells infected with FluA or RSV. The therapeutic index (TI) was >12 for Flu A and about 9.6 for RSV (FIGS. 10A and 10B, Table 2).

TABLE 2
SLC1021 Cytoprotection Assay

| | EC50 | TC50 | |
|---|---|---|---|
| Virus Strain/Cells | (μg/mL) | (μg/mL) | TI |
| SAR-CoV2_{USA/WA1/2020}/VeroE6 cells | >470 | 470 | . . . |
| Flu_{APR834}/RPMI2650 cells | <10 | 120 | >12 |
| RSV_{A2}/PRMI2650 cells | 70 | 670 | 9.57 |
| Zika_{PRVABC59}/Huh7 cells | 130 | 400 | 3.08 |
| DENV2_{New Guinea}/Huh7 cells | >300 | 300 | . . . |

TI: Therapeutic Index

### Example 7

### Cytotoxicity Effect of SLC1021 on Tumor Cells

The cytotoxicity of SLC1021 on cancer cells was investigated. Jurkat, HL60, THP1, MCF7 and LNCaP cell-lines were used in the cytotoxicity assays. In addition, the redox state of Jurkat cells and primary human T-cells after SLC1021 exposure was evaluated. Treated red lettuce extract (SLC1021) was prepared using the methods as described in Examples 1 and 2. Major polyphenols were characterized and quantified with the LC-MS analysis.

Method: Jurkat, HL60, THP1, MCF7 and LNCaP cell-lines were cultured according to ATCC instructions. The viability of the cells was assessed by MTS (Promega, G111A) and PMS (Sigma, P9625) assays. One day prior to assay, MCF7 and LNCaP (adherent cells) were trypsinized and washed with culture medium. The cells were resuspended in 10% fetal bovine serum (FBS) medium and seeded (2×10⁴ cells/well) in a ninety-six-well plate (Sarstedt) for overnight. On the day of SLC1021 treatment, the culture medium was carefully removed and replaced with 1% FBS medium. The remaining suspension cell-lines were washed, resuspended in 1% FBS medium, and seeded (2×10⁴ cells/well) in a ninety-six-well plate. All cells were then treated with SLC1021 for 48 h (with total volume of 100 μl per well) at 37° C. in a cell culture incubator containing 5% CO₂. Thereafter, 25 μl of MTS solution was added to each well and incubated at 37° C. for 2 h. Finally, spectrophotometric absorbance was recorded at 490 nm using a microplate reader (SpectraMax i3X, Molecular Devices). The toxic concentration causing 50% cell death (TC₅₀; μg/mL) was determined by GraphPad Prism (GraphPad Software).

Intracellular reactive oxygen species (ROS) production was monitored using an oxidant sensitive fluorescent probe DCF-DA (OZBiosciences, ROS0300). Primary human T cells were isolated from human peripheral blood mononuclear cells (Stemcell, 70025.1) using a CD3 positive cell isolation kit (Stemcell, 17951). The primary T cells were then activated with anti-CD3 antibodies (R&D systems, MAB100) at 3 μg/mL for 72 h. Activated T cells were then expanded in culture with human IL-2 (Sigma, SRP3085) at 50 ng/mL for 7 days before applying to assay. Jurkat cells and primary T-cells were seeded in 96-well plate (1×10⁵ cells/well) and treated with SLC1021 from 6.9 to 556.7 μg/mL for 24 h in medium containing 1% FBS. The cells were harvested and stained with 2 μM DCF-DA for 30 min according to manufacturer's protocol. ROS production was detected by flow cytometry.

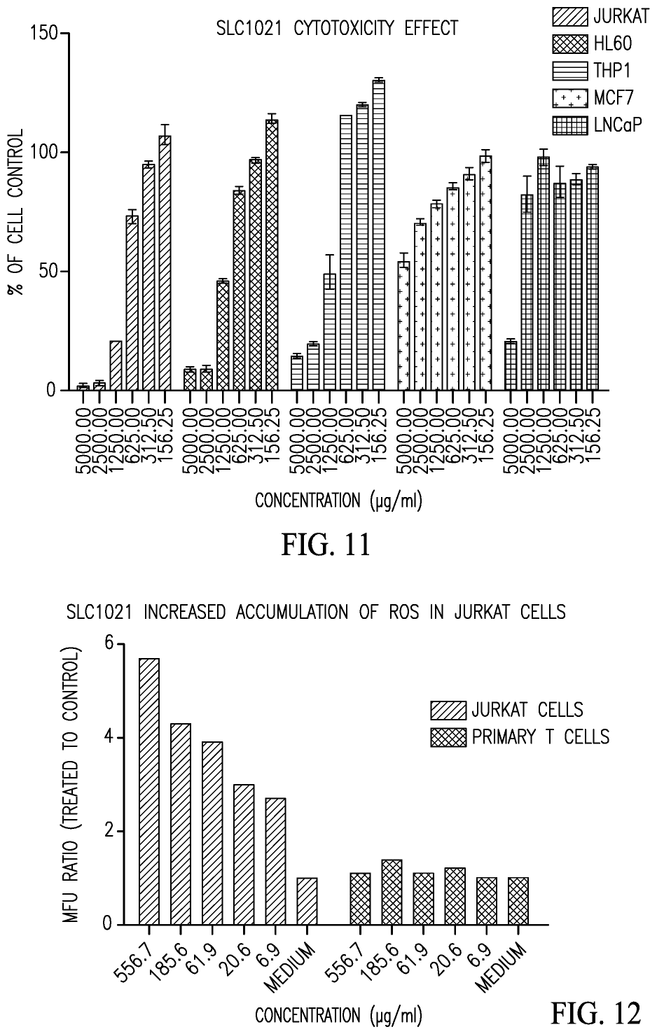

Results: The MTS assay showed the SLC1021 extract had a cytotoxic effect on the tested cell-lines in a concentration dependent manner. The following TC₅₀ values were calculated for each cell line: Jurkat, 799.8 μg/mL; HL60, 1004.6 μg/mL; THP1, 1039.9 μg/mL, and LNCaP, 2766.9 μg/mL (FIG. 11).

Jurkat cells treated with 6.9 μg/mL for 24 h displayed increased ROS content compared to the untreated control Jurkat cells (FIG. 12). Increased concentration of SLC1021 resulted in significantly increased ROS level in Jurkat cells. Jurkat cells treated with SLC1021 at 556.7 μg/mL for 24 h had the highest ROS level and the cytotoxicity observed from the cytotoxicity assay implied that the death of Jurkat cells was associated with disruption of intracellular redox balance caused by increased ROS level and decreased antioxidant capacity. Such disruption of intracellular redox reaction is not observed with primary T-cells. These data indicate the potential anti-cancer mechanism of SLC1021 without having any noticeable effect on human primary T-cells.

### Example 8

### Cytotoxicity Effects of SLC1021, SLC1021-B and Major Polyphenol Components of SLC1021 on Tumor Cells

In order to evaluate the biological effects of SLC1021 (extract of treated lettuce) and SLC1021-B (extract of untreated lettuce, baseline polyphenol content), the cytotoxicity effects of SLC1021 and SLC-1021-B on tumor cells were performed. In order to compare biological activities of SLC1021 with major individual polyphenol components of SLC1021, the cytotoxicity effect of SLC1021 and a selection of major individual polyphenol components on tumor cells were carried out. Treated red lettuce extract (SLC1021) with significantly enhanced health beneficial polyphenols and untreated lettuce extract (SLC1021-B) with baseline polyphenol contents were prepared using the methods as described in Examples 1 and 2. Major polyphenols were characterized and quantified with the LC-MS analysis.

Methods: Jurkat, THP1, and MCF7 cell-lines were cultured according to ATCC instructions. The viability of the cells was assessed by MTS and PMS assays. One day prior to assay, MCF7 cells were trypsinized and washed with culture medium. The cells were resuspended in 10% FBS medium and seeded (2×10⁴ cells/well) in a ninety-six-well plate for overnight before MTS assay. On the day of cell treatment, the culture medium was carefully removed and replaced with 1% FBS medium. The suspension cell-lines were washed, resuspended in 1% FBS medium, and seeded (2×10⁴ cells/well) in a ninety-six-well plate. All the cells were then treated with SLC1021, SLC1021-B, chicoric acid, 4-CQA, neochlorogenic acid, or cyanidin 3-galactoside for 48 h (with total volume of 100 μl per well) at 37° C. in cell culture incubator containing 5% CO₂. Thereafter, 25 μl of MTS solution was added to each well and incubated at 37° C. for 2 h. The absorbance was recorded at 490 nm using a microplate reader. TC₅₀ (μg/mL) was determined by GraphPad Prism (GraphPad Software).

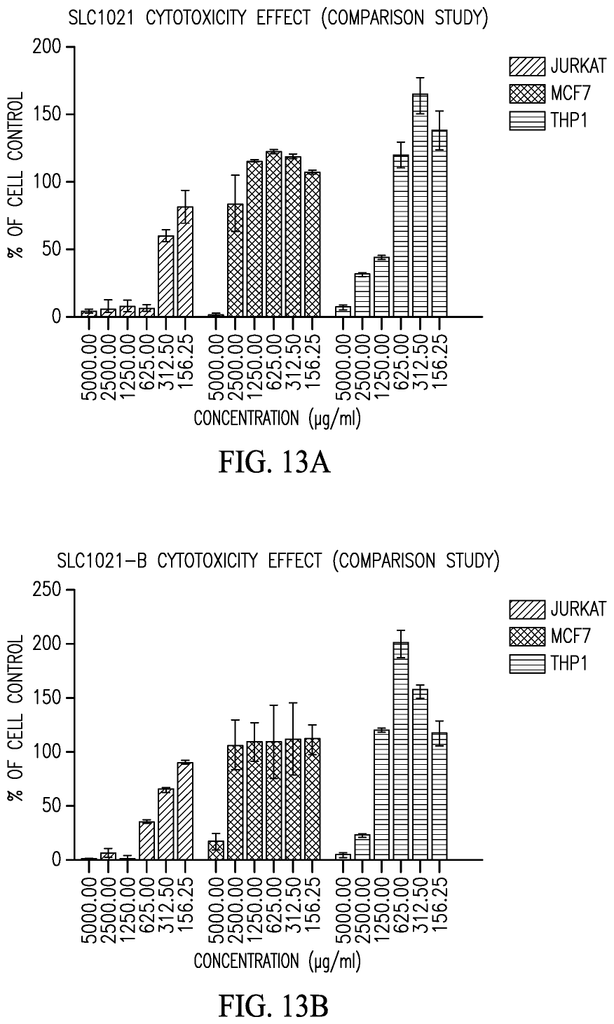

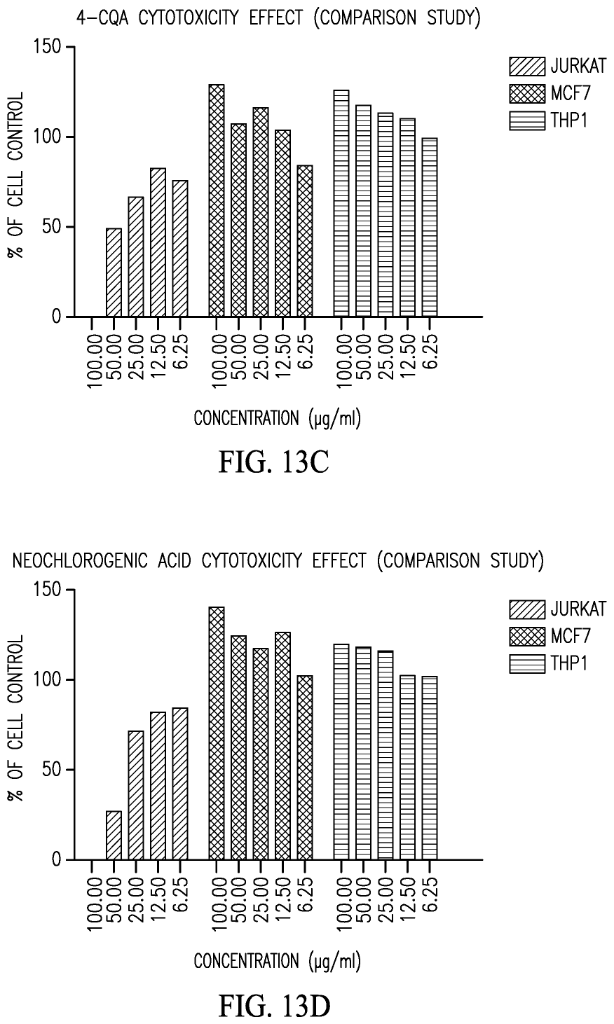

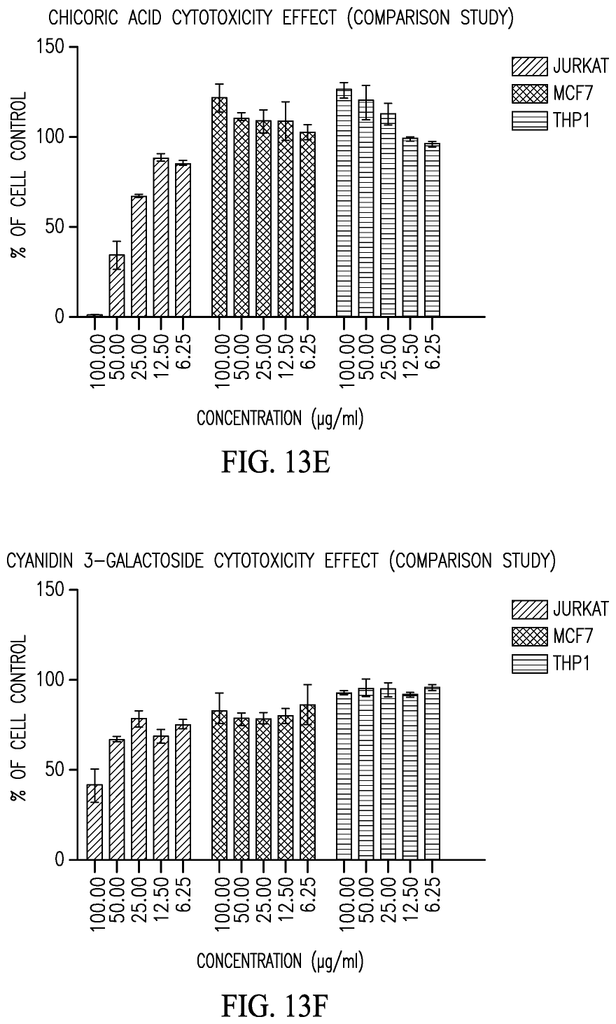

Result: The cytotoxicity effect of SLC1021 was compared with SLC1021-B and individual components (chicoric acid, 4-CQA, neochlorogenic acid, and cyanidin 3-galactoside) on cancer cells. Cells were incubated with equivalent concentrations (w/w) for 48 h. The MTS assay showed consistent SLC1021 cytotoxic effect on the tested cell-lines in concentration dependent manner (FIG. 13A). SLC1021 was more cytotoxic than SLC1021-B on the tested cell-lines (FIGS. 13A and 1321B). Chicoric acid, 4-CQA, neochlorogenic acid, and cyanidin 3-galactoside demonstrated cytotoxicity activity towards Jurkat cells (FIGS. 13C, 13D, 13E and 13F), but was lower than SLC1021. Chicoric acid, 4-CQA, neochlorogenic acid, and cyanidin 3-galactoside did not appear to be individually cytotoxic towards THP1 and MCF7 cell-lines. Table 3 showed the cytotoxic effect of SLC1021, SLC1021-B, chicoric acid, 4-CQA, neochlorogenic acid, and cyanidin 3-galactoside on 3 cell-lines. Overall, SLC1021 TC₅₀ is lower than SLC1021-B, and SLC1021 showed superior cytotoxic effects against multiple cancer cells compared to SLC1021-B, chicoric acid, 4-CQA, neochlorogenic acid, and cyanidin 3-galactoside.

TABLE 3
TC₅₀ of SLC1021, SLC1021-B, 4-CQA, neochlorogenic
acid, chicoric acid, and cyanidin 3-galactoside

| | Jurkat, TC₅₀ | MCF7, TC₅₀ | THP1, TC₅₀ |
|---|---|---|---|
| Treatment | (μg/mL) | (μg/mL) | (μg/mL) |
| SLC1021 | 355.6 | 2729 | 1086 |
| SLC1021-B | 459.2 | 4864 | 2399 |
| 4-CQA | 49.9 | — | — |
| Neochlorogenic acid | 42.4 | — | — |
| Chicoric acid | 48.9 | — | — |
| Cyanidin-3 galactoside | 53.5 | — | — |

TC₅₀: Toxic concentration that caused 50% cell death

### Example 9

### Antiinflammatory Effects of SLC1021, SLC1021-B and Individual Polyphenol Components of SLC1021

In order to investigate the anti-inflammatory effect of SLC1021, SLC1021-B, and individual phenolic bioactive components of interest found in SLC1021 (i.e., 4-CQA, neochlorogenic acid, chicoric acid, and cyanidin 3-galactoside), LPS was used to stimulate the release of IL-6 and TNF-α in the PMA-differentiated THP1 macrophage cells to mimic inflammatory environment.

Methods: PMA-differentiated THP1 macrophages were used to evaluate the anti-inflammatory effect of SLC1021 and SLC1021-B. THP1 monocytes were differentiated to macrophages using phorbol 12-myristate 13-acetate (PMA, Sigma, P1585). The

THP1 cells were resuspended in 10% fetal bovine serum (FBS) medium and seeded (1×10⁵ cells/well, 100 μl volume) in a ninety-six-well plate in the presence of 25 nM PMA for 2 days. On the day of assay, the culture medium was removed and replaced with 1% FBS medium containing 500 ng/mL IFN-γ (Sino, GMP-11725-HNAS) (100 μl per well). The cells were treated with different concentrations of SLC1021 or SLC1021-B (0.02, 0.06, 0.19, 0.56, 1.67 and 5 mg/mL) for 2 h and then with LPS (Sigma, L2630) for an additional 48 h (total volume of 200 μl per well). Macrophages exposed to LPS but not treated with SLC1021 or SCL1021-B were used as a control (untreated cells). The culture supernatant was collected from each well and replaced with 100 μl of fresh 1% FBS medium. For the measurement of percent (%) of cell control, 25 μl of MTS solution was added to each well and incubated at 37° C. for 2 h. The absorbance was recorded at 490 nm using a microplate reader (SpectraMax i3X, Molecular Devices). 50 μl of cultured supernatant was used for measuring TNF-α and IL6 concentration. TNF-α was measured using human TNF-α DuoSet ELISA kit (R&D systems, DY210-05) and IL6 was determined using human IL6 DuoSet ELISA kit (R&D systems, DY206-05) according to the manufacturer's instructions. TC₅₀/EC₅₀ (μg/ml) was determined by GraphPad Prism (GraphPad Software).

THP1 monocytes were differentiated to macrophages as previously described. On the day of assay, the culture medium was removed and replaced with 1% FBS medium containing 500 ng/ml IFN-γ (100 μl per well). The cells were pre-treated with 4-CQA, neochlorogenic acid, chicoric acid, and cyanidin 3-galactoside at 1.23, 3.7, 11.11, 33.33 and 100 μg/mL for 2 h. Untreated cells were used as the control. After incubation, cell cultures were then stimulated with LPS for an additional 48 h (total volume of 200 μL per well). The culture supernatant was collected from each well and replaced with 100 μL of fresh 1% FBS medium. For the measurement of % of cell control, 25 μl of MTS solution was added to each well and incubated at 37° C. for 2 h. The absorbance was recorded at 490 nm using a microplate reader. 50 μl of cultured supernatant was used for measuring TNF-α and IL6 concentration. TNF-α was measured using human TNF-α DuoSet ELISA kit and IL6 was determined using human IL6 DuoSet ELISA kit according to the manufacturer's instructions.

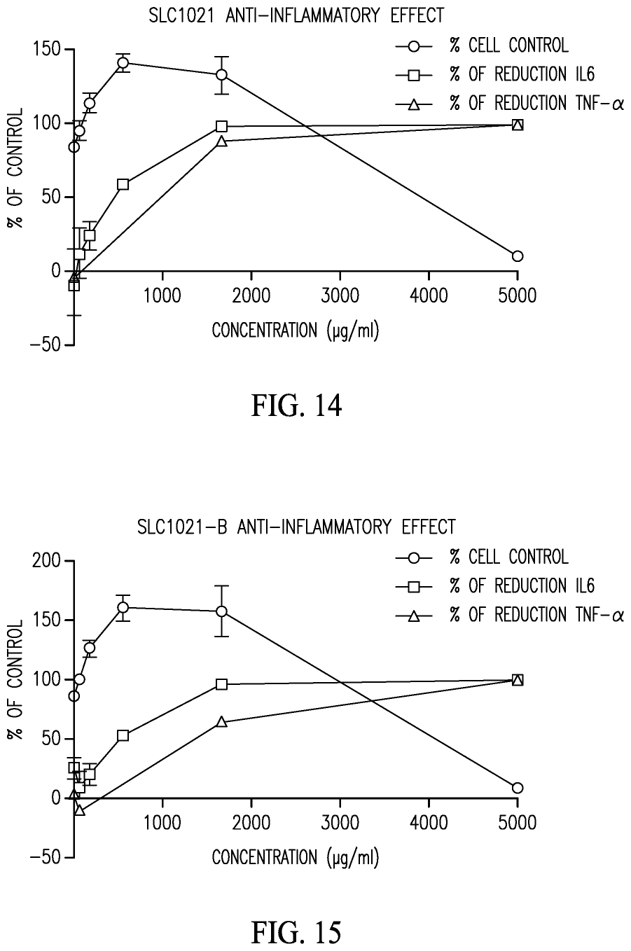

Results: The anti-inflammatory effect of SLC1021 was investigated. LPS was used to stimulate the release of IL-6 and TNF-α in the PMA-differentiated THP1 macrophage cells to mimic inflammatory environment (FIG. 14). LPS enhanced production of IL-6 and TNF-α (data not shown) for 48 h, and pre-treatment with various concentrations (0.02, 0.06, 0.19, 0.56, 1.67 and 5 mg/mL) of SLC1021 prior to LPS challenge reduced secretion of the pro-inflammatory cytokines. Anti-inflammatory effect on the macrophage is concentration dependent and the effect is not related to cytotoxicity at concentration below 1.67 mg/ml. Overall, the experiment demonstrates the anti-inflammatory effects of SLC1021 on human macrophages.

The comparison of anti-inflammatory effect of SLC1021B was also carried out (FIG. 15). The conditions and treatments were the same as SLC1021. Anti-inflammatory effect on the macrophage is concentration dependent and the effect was not related to cytotoxicity at concentration below 1.67 mg/mL. At 1.67 mg/ml the % of TNF-α reduction is significantly lower than SLC1021. Table 4 showed the anti-inflammatory effect of SLC1021 and SLC1021-B. The overall therapeutic index (TI) for SL1021 was higher than SLC1021-B. In another word, the anti-inflammatory effects of SLC1021-B is lower than SLC1021 on human macrophages.

TABLE 4
Effects of SLC1021 and SLC1021-B on PMA-differentiated
THP1 macrophage anti-inflammatory assay

| | IL6, EC₅₀ | TNF-α, EC₅₀ | TC₅₀ | IL6, | TNF-α, |
|---|---|---|---|---|---|
| Treatment | (μg/mL) | (μg/mL) | (μg/mL) | TI | TI |
| SLC1021 | 383 | 887 | 3967 | 10.3 | 4.47 |
| SLC1021-B | 567 | 1371 | 4092 | 7.2 | 2.98 |

TI: Therapeutic Index
TC₅₀: Toxic concentration that caused 50% cell death

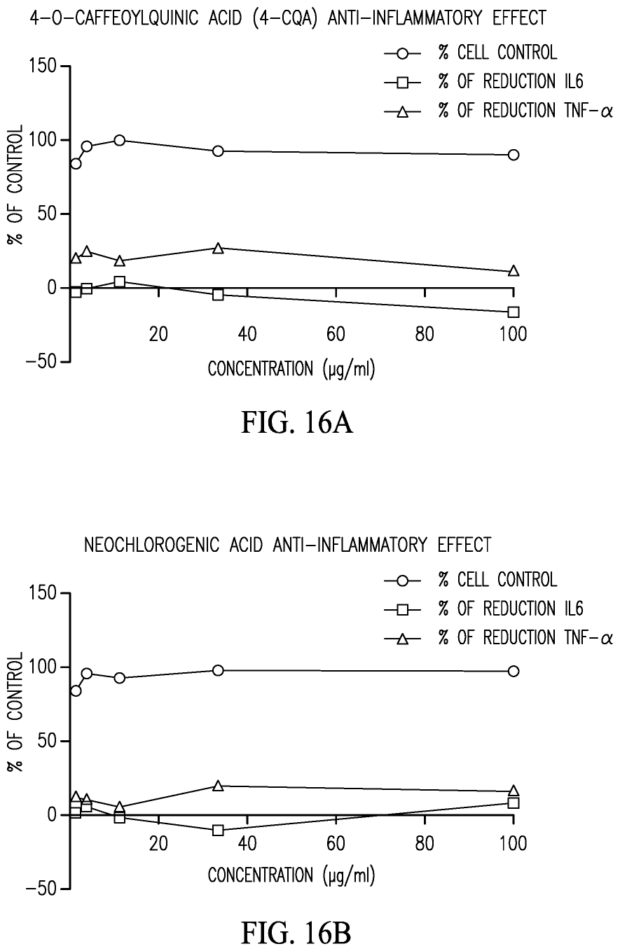

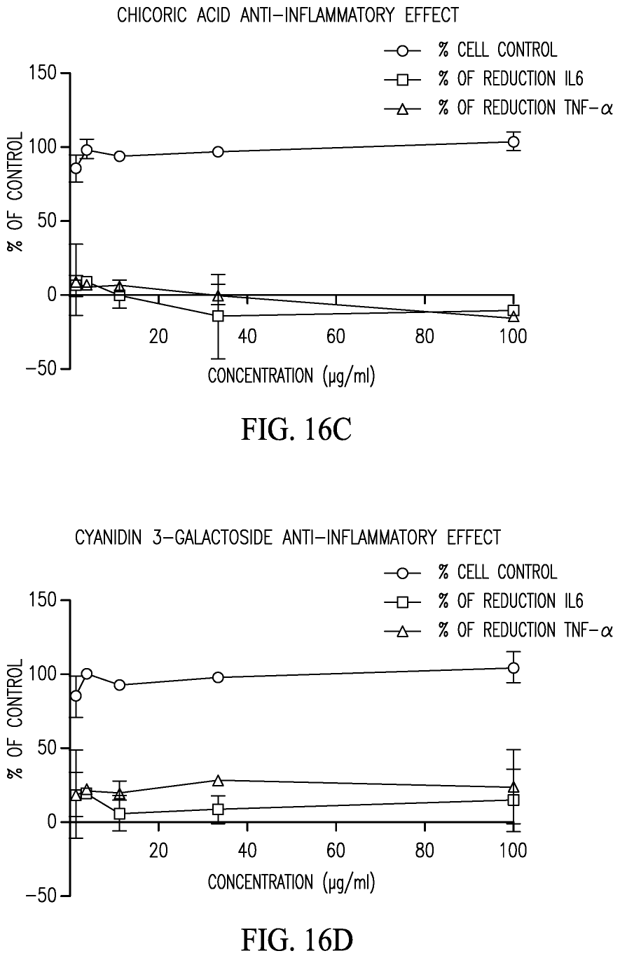

As described herein, chlorogenic acids, chicoric acid, quercetin derivatives and anthocyanins have been detected major bioactive compounds found in the SLC1021 lettuce extract. Chlorogenic acids, chicoric acid, and anthocyanins each make up about 2% (w/w) of SLC1021 and quercetin is around 3.5% (w/w). The anti-inflammatory effects of 4-CQA, neochlorogenic acid, chicoric acid, and cyanidin 3-galactoside was investigated using PMA-differentiated THP1 macrophage cells stimulated with LPS. Quercetin data was excluded due to its color interference on cytotoxicity evaluation by MTS staining. LPS stimulated production of IL-6 and TNF-α for 48 h. Macrophages were pre-treated with 1.23, 3.7, 11.11, 33.33 and 100 μg/mL of 4-CQA, neochlorogenic acid, chicoric acid, and cyanidin 3-galactoside and then treated with LPS. Individually, 4-CQA, neochlorogenic acid, chicoric acid, and cyanidin 3-galactoside showed minimal effects on secretion of the pro-inflammatory cytokines IL-6 and TNF-α (FIGS. 16A-D). The components were not cytotoxic to PMA-differentiated THP1 macrophages. In summary, the experiment demonstrated the potential synergistic anti-inflammatory effect of various components within SLC1021 on human macrophages.

### Example 10

### Antioxidant Effects of SLC1021, SLC1021-B and Individual Polyphenol Components of SLC1021

In order to investigate the anti-oxidant effect of SLC1021, SLC1021 B, and individual polyphenol bioactive components of SLC1021, LPS was used to stimulate the release of nitric oxide (NO) in the PMA-differentiated THP1 macrophage cells to mimic inflammatory environment.

Methods: Assay to test nitric oxide (NO) production used PMA-differentiated THP1 macrophages and was set up as described above 42.5 μL of cultured supernatant was used for measuring nitric oxide (nitrite). Total nitrite was measured using nitric oxide colorimetric assay kit (BioVision, K262-200) according to the manufacturer's instructions. TC₅₀/EC₅₀ (μg/mL) was determined by GraphPad Prism (GraphPad Software).

For the evaluation of major individual components in SLC1021, THP1 monocytes were differentiated to macrophages as previously described. On the day of assay, the culture medium was removed and replaced with 1% FBS medium containing 500 ng/mL IFN-γ (100 μl per well). The cells were pre-treated with 4-CQA, neochlorogenic acid, chicoric acid, and cyanidin 3-galactoside at 1.23, 3.7, 11.11, 33.33 and 100 μg/mL for 2 h. Untreated cells were used as the control. After incubation, cell cultures were then stimulated with LPS for an additional 48 h (total volume of 200 μl per well). The culture supernatant was collected from each well and replaced with 100 μl of fresh 1% FBS medium. For the measurement of % of cell control, 25 μl of MTS solution was added to each well and incubated at 37° C. for 2 h. The absorbance was recorded at 490 nm using a microplate reader. 42.5 μL of cultured supernatant was used for measuring nitrite concentration. Total nitrite was measured using nitric oxide colorimetric assay kit according to the manufacturer's instructions. EC₅₀ (μg/mL) was determined by GraphPad Prism (GraphPad Software).

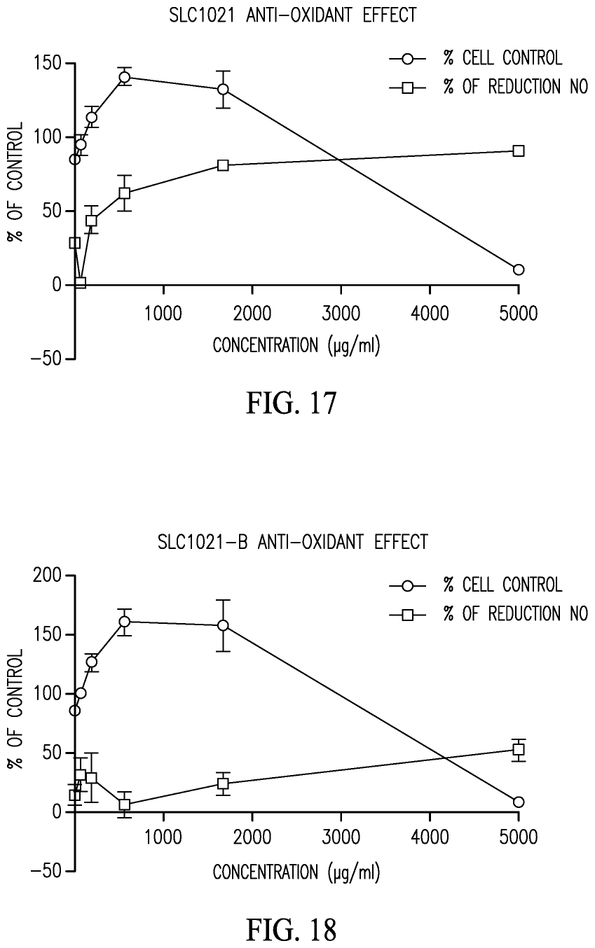

Results: The anti-oxidant effect of SLC1021 was concentration dependent and the effect was not related to cytotoxicity at concentration below 1.67 mg/mL (FIG. 17). Compared to SLC1021, the anti-oxidant effect of SLC1021-B was significantly lower (FIG. 18).

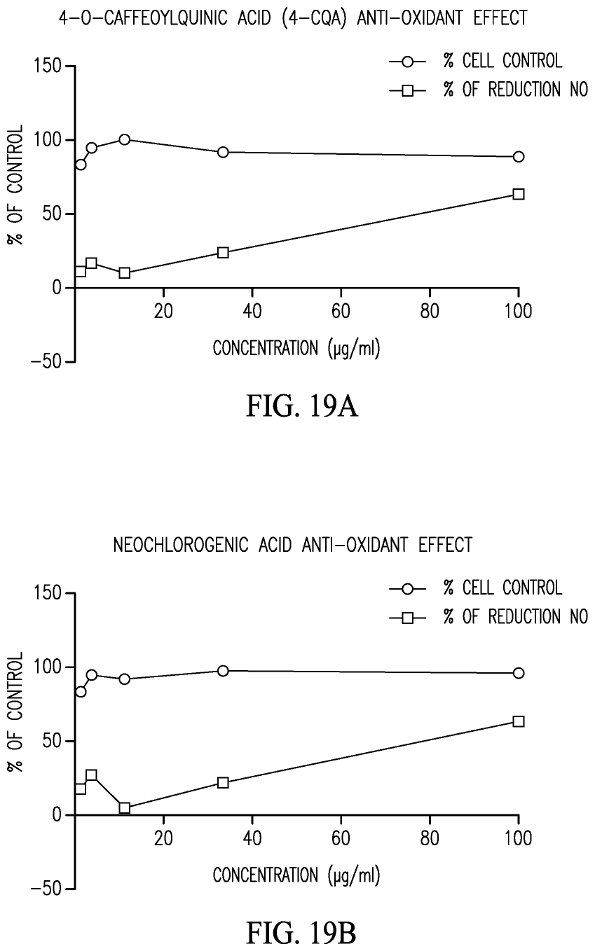

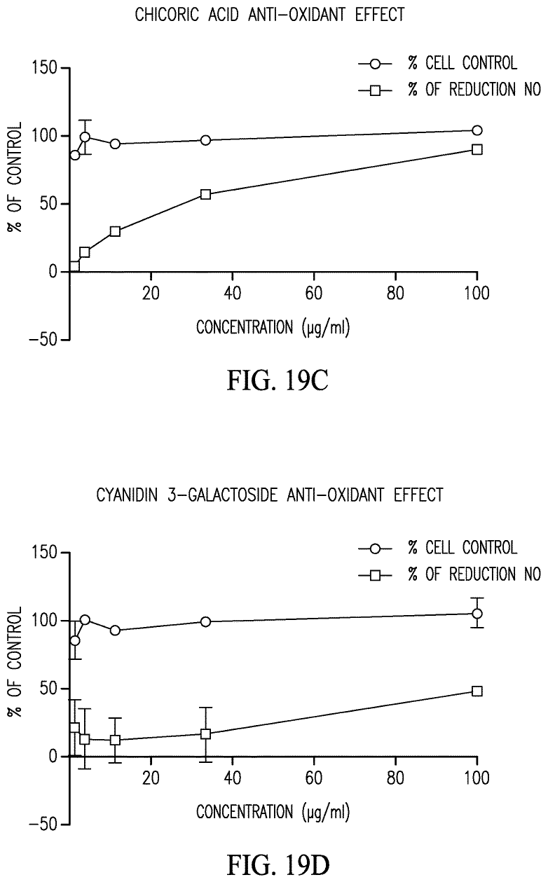

FIGS. 19A-19D shows the effect that 4-CQA, neochlorogenic acid, chicoric acid, and cyanidin 3-galactoside had on nitric oxide production. Table 5 summarizes the anti-oxidant effect of SLC1021, SLC1021-B, 4-CQA, neochlorogenic acid, chicoric acid, and cyanidin 3-galactoside. The overall therapeutic index (TI) for SL1021 was higher than SLC1021-B. In another word, the anti-oxidant effects of SLC1021-B are lower than SLC1021 on human macrophages. In summary, the experiment demonstrated the potential synergistic therapeutic effect of various components within SLC1021 on human macrophages, independent from anti-oxidant effects.

TABLE 5
Effect of SLC1021, SLC1021B, and 4-CQA, Neochlorogenic
Acid, Chicoric Acid, and Cyanidin 3-Galactoside
on THP1 Macrophage Anti-oxidant Assay

| | NO, EC50 | TC₅₀ | |
|---|---|---|---|
| Treatment | (μg/mL) | (μg/mL) | NO, TI |
| SLC1021 | 345.1 | 3967 | 11.50 |
| SLC1021-B | 2032.4 | 4092 | 2.01 |
| 4-CQA | 71.9 | | |
| Neochlorogenic acid | 80.5 | | |
| Chicoric acid | 25.3 | | |
| Cyanidin 3-galactoside | 98.4 | | |

TI: Therapeutic Index
TC₅₀: Toxic concentration that caused 50% cell death

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 63/154,529, filed on Feb. 26, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A system for biosynthesis of polyphenols in lettuce, comprising at least one eustressor/elicitor, or a homologue, isomer or derivative thereof, that increase the production of polyphenols in a lettuce plant.

2. A method for biosynthesis of polyphenols in lettuce, comprising administering a system according to claim 1 to a lettuce plant, thereby increasing the production of polyphenols in lettuce.

3. The system of claim 1, wherein the at least one eustressor/elicitor is an abiotic eustressor/elicitor.

4. The system of claim 3 wherein the abiotic eustressor/elicitor is selected from: auxins, cytokinins (CKs), gibberellins (GAs), ethylene, brassinosteroids, jasmonates (JAs), strigolactones (SLs), salicylic acid (SA), arachidonic acid (AA), 5-aminolevumic acid (5-ALA), oxalic acid, and any homologues or isomers or derivatives, synthetic analogues, or any combination or mixture thereof.

5. (canceled)

6. The system of claim 3, wherein the at least one abiotic eustressor/elicitor is selected from: indole-3-acetic acid (IAA), indole-3-acetonitril (IAN), indole-3-acetaldehyde (IAc), ethylindoeacetate, indole-3-pyruvic acid (IPyA), indole-3-butyric acid (IBA), indole-3-propionic acid (IPA), indazole-3-acetic acid, chlorophenoxypropionic acids, naphthalene acetic acid (NAA), phenoxy acetic acid (PAA), 2,4-dichlorophenoxy acetic acid (2,4-D), 2,4,5-trichlorophenoxy acetic acid (2,4,5-T), naphthalene acetamide (NAAM), 2-napthoxyacetic acid (NOA), 2,3,5-triodobenzoic acid (TIBA), thianaphthen-3-propionic acid (IPA), ribosylzeatin, zeatin, isopentinyladenine, dihydrozeatin, 6-benzyl amino purine, 6-phenyl amino purine, kinetin, N-benzyl-9-(2-tetrahydropyranyl) adenine (BPA), diphenylurea, thidiazuron, benzimidazole, adenine, 6-(2-thenylamino) purine, GA, GA4, GA7, GA3, ethylene, ethephon, ethrel, dolicholide, 28-homodolicholide, castasterone, dolichosterone, 28-homodolichosterone, typhasterol, jasmonic acid, methyl dihydrojasmonate, dihydrojasmonic acid, methyl jasmonate (MJ), strigol, orobanchol, GR24, arachidonic acid (AA), salicylic acid (SA), or any combination or mixture thereof.

7.-8. (canceled)

9. The system of claim 1, wherein the system comprises the eustressor/elicitor selected from: indole-3-acetic acid (IAA), naphthalene acetic acid (NAA), oxalic acid, benzothiadiazole (BTH), 2,4-dichlorophenoxy acetic acid (2,4-D), arachidonic acid (AA), salicylic acid (SA), and/or methyl jasmonate (MJ), wherein each elicitor is independently at a concentration of 5 μM, 10 μM, 15 μM, 45 μM, or 90 μM.

10. The system of claim 1, wherein the at least one eustressor/elicitor is a biotic eustressor/elicitor (bio-stimulant) selected from: lipopolysaccharides, pectin and cellulose (cell walls), chitosan, chitin and glucans, alginate, Burdock fructooligosaccharide (BFO), Arabic gum, yeast extract, seaweed extract, humic and fulvic acid, one or more botanical extracts from Reynoutria Sachalinensis, Reynoutria japonica extract, moringa leaf, cregano, sugar beet, linseed, St. John's wort (Hypericum perforatum L.; herb), giant goldenrod (Solidago gigantean Ait.; leaf), common dandelion (Taraxacum officinale (L.) Weber ex F. H. Wigg; flower, leaf), red clover (Trifolium pretense L.; flower), nettle (Urtica dioica L.; leaf), valerian (Valeriana officinalis L.; root), garlic, Chinese chive, licorice root, red grape skin, blueberry fruits, hawthorn leaves, common mugwort, olive leaves, pomegranate leaves, common guava leaves, borage leaves and flowers, cultivated tobacco leaves, bael leaves, fig tree leaves, hina tree leaves, Chinese chaste tree leaves, wild celery leaves, French oak, maize grain, rosemary, palm pollen grains, alfalfa plant, galacturonides, gluronate, mannan, mannuronate, cellulase, cryptogein, glycoproteins, harpin protein (HP), glycoprotein, oligandrin, pectolyase, fish protein, hydrolysates, lactoferrin, fungal spores, mycelia cell wall, microbial wall, coronatine, and oregano extract.

11. (canceled)

12. The system of claim 10, wherein the system comprises the eustressor/elicitor at a concentration of 10 mg to 5000 mg/L.

13. The system of claim 10, wherein the system comprises the biotic eustressor/elicitor Harpin protein (HP), Burdock fructooligosaccharide (BFO), and/or chitosan at a concentration of 30 mg/L, 60 mg/L, or 120 mg/L.

14. The system of claim 1, wherein the polyphenol is chlorogenic acid/derivatives, water-soluble quercetin derivatives, and anthocyanins.

15. The system of claim 14, wherein:
the chlorogenic acid is 3-O-caffeoylquinic acid (3-CQA), 4-O-caffeoylquinic acid (4-CQA), and/or 5-O-caffeoylquinic acid (5-CQA), chicoric acid, 3,4-dicaffeoylquinic acid (3,4-diCQA);
(ii) the water-soluble quercetin derivative is quercetin-3-O-glucoside (Q3G) and/or quercetin-3-O-malonylglucoside (Q3MG), and/or
(iii) the anthocyanin is cyaniding 3-malonyl-glucoside and/or cyandidin-3-O-glucoside.

16.-70. (canceled)

71. The method of claim 2, wherein the lettuce plant is a red leaf lettuce cultivar is-selected from Lollo Rossa, New Red Fire Lettuce, Red Sails Lettuce, Redina Lettuce, Galactic Lettuce, Batavian lettuce, Annapolis, Lettuce, Hongjil Lettuce, Red Fire Lettuce, Jinluck Lettuce, Dazzler Lettuce, Seoul Red Lettuce, Revolution Lettuce, Cherokee Lettuce, Valerial Lettuce, OOC 1441 Lettuce, Impuls Lettuce, Red Mist Lettuce, Red Salad Bowl Lettuce, Red Tide Lettuce, Bellevue Lettuce, Outredgeous Lettuce, Pomegranate Crunch Lettuce, Vulcan Lettuce, Cantarix Lettuce, Breen Lettuce, Rouge D'Hiver Lettuce, Oscarde Lettuce, Blade Lettuce, Spock Lettuce, Edox Lettuce, Fortress Lettuce, Stanford Lettuce, Scaramanga Lettuce, Rutgers Scarlet Lettuce, and Benito Lettuce.

72. (canceled)

73. The method of claim 2, wherein the at least one eustressor/elicitor is selected from one or more of:
(i) an abiotic eustressor/elicitor selected from: auxins, cytokinins (CKs), gibberellins (GAs), ethylene, brassinosteroids, jasmonates (JAs), strigolactones (SLs), salicylic acid (SA), arachidonic acid (AA), 5-aminolevumic acid (5-ALA), oxalic acid, and any homologues or isomers or derivatives, synthetic analogues, or any combination or mixture thereof, and/or
(ii) an abiotic eustressor/elicitor is selected from: indole-3-acetic acid (IAA), indole-3-acetonitril (IAN), indole-3-acetaldehyde (IAc), ethylindoeacetate, indole-3-pyruvic acid (IPyA), indole-3-butyric acid (IBA), indole-3-propionic acid (IPA), indazole-3-acetic acid, chlorophenoxypropionic acids, naphthalene acetic acid (NAA), phenoxy acetic acid (PAA), 2,4-dichlorophenoxy acetic acid (2,4-D), 2,4,5-trichlorophenoxy acetic acid (2,4,5-T), naphthalene acetamide (NAAM), 2-napthoxyacetic acid (NOA), 2,3,5-triodobenzoic acid (TIBA), thianaphthen-3-propionic acid (IPA), ribosylzeatin, zeatin, isopentinyladenine, dihydrozeatin, 6-benzyl amino purine, 6-phenyl amino purine, kinetin, N-benzyl-9-(2-tetrahydropyranyl) adenine (BPA), diphenylurea, thidiazuron, benzimidazole, adenine, 6-(2-thenylamino) purine, GA, GA4, GA7, GA3, ethylene, ethephon, ethrel, dolicholide, 28-homodolicholide, castasterone, dolichosterone, 28-homodolichosterone, typhasterol, jasmonic acid, methyl dihydrojasmonate, dihydrojasmonic acid, methyl jasmonate (MJ), strigol, orobanchol, GR24, arachidonic acid (AA), salicylic acid (SA), or any combination or mixture thereof; and/or
(iii) a biotic eustressor/elicitor (bio-stimulant) selected from: lipopolysaccharides, pectin and cellulose (cell walls), chitosan, chitin and glucans, alginate, Burdock fructooligosaccharide (BFO), Arabic gum, yeast extract, seaweed extract, humic and fulvic acid, one or more botanical extracts from Reynoutria Sachalinensis, Reynoutria japonica extract, moringa leaf, cregano, sugar beet, linseed, St. John's wort (Hypericum perforatum L.; herb), giant goldenrod (Solidago gigantean Ait.; leaf), common dandelion (Taraxacum officinale (L.) Weber ex F. H. Wigg; flower, leaf), red clover (Trifolium pretense L.; flower), nettle (Urtica dioica L.; leaf), valerian (Valeriana officinalis L.; root), garlic, Chinese chive, licorice root, red grape skin, blueberry fruits, hawthorn leaves, common mugwort, olive leaves, pomegranate leaves, common guava leaves, borage leaves and flowers, cultivated tobacco leaves, bael leaves, fig tree leaves, hina tree leaves, Chinese chaste tree leaves, wild celery leaves, French oak, maize grain, rosemary, palm pollen grains, alfalfa plant, galacturonides, gluronate, mannan, mannuronate, cellulase, cryptogein, glycoproteins, harpin protein (HP), glycoprotein, oligandrin, pectolyase, fish protein, hydrolysates, lactoferrin, fungal spores, mycelia cell wall, microbial wall, coronatine, oregano extract, or any combination thereof.

74. An extract of the lettuce of claim 2, comprising an increased production of polyphenols or derivatives thereof, relative to a control extract.

75. The extract of claim 74, wherein the extract is red lettuce extract SLC1021.

76. The extract of claim 74, wherein the extract comprises water and ethanol and lettuce components that are soluble therein.

77. A method of making a lettuce extract of claim 74, comprising mixing a lettuce sample with a solvent and separating the liquid phase from the solid phase.

78. The method of claim 77, wherein the solvent is ethanol.

79. (canceled)

80. The method of claim 77, wherein the ratio of lettuce to solvent (g/mL) is 1:10, 1:5, 2:5, 3:5, 4:5, or 1:1.

81. A food product containing the lettuce or parts thereof according to claim 2.

82.-85. (canceled)

86. A method for treating a respiratory infection by coronavirus comprising administering an effective amount of the extract of claim 74 to a patient infected with a coronavirus, and wherein the coronavirus is inhibited by inhibition of 3-chymotrypsin-like protease (3CL^{pro}) activity, inhibition of RNA-dependent RNA polymerase (RdRp) activity, inhibition of RNA helicase and triphosphatase (nsp13) activity, inhibition of binding of a Spike protein to ACE2, or any combination thereof.

87.-89. (canceled)

90. The method of claim 86, wherein the coronavirus is a severe acute respiratory syndrome coronavirus 2 (SARS-CoV-2).

91.-92. (canceled)

93. A method for treating an influenza A (Flu A) infection, comprising administering an effective amount of the extract of claim 74 to a patient infected with Flu A.

94. (canceled)

95. A method for treating a respiratory syncytial virus (RSV) infection, comprising administering an effective amount of the extract of claim 74 to a patient infected with RSV.

96. (canceled)

97. A method for treating a Zika virus infection, comprising administering an effective amount of the extract of claim 74 to a patient infected with Zika virus.

98. A method for treating a Dengue (DENV2) virus infection, comprising administering an effective amount of the extract of claim 74 to a patient infected with DENV2.

99. (canceled)

100. A method for treating a cancer, comprising administering an effective amount the extract of claim 74 to a patient in need thereof.

101. (canceled)

102. A method for treating an inflammatory condition or disease, comprising administering an effective amount of the extract of claim 74 to a patient in need thereof.

103.-107. (canceled)
